(19) United States
(12) Patent Application Publication
(10) Pub. No.: US 2010/0076037 A1
(43) Pub. Date: Mar. 25, 2010

(54) Methods of Treating Neuropathic Pain with Agonists of PPAR-gamma

(76) Inventors: Lillian W. Chiang, Princeton, NJ (US); Tage Honore, Princeton, NJ (US)

Correspondence Address: AESTUS THERAPEUTICS INC., 675 US HIGHWAY ONE, NORTH BRUNSWICK, NJ, 08902, US

(21) Appl. No.: 12/513,234

(22) PCT Filed: Nov. 1, 2007
(86) PCT No.: PCT/US07/83326

§ 371 Date: May 1, 2009

Related U.S. Application Data
(60) Provisional application No. 60/864,095, filed on Nov. 2, 2006.

(51) Int. Cl. A61K 31/255; A61K 31/421; A61K 31/192; A61K 31/335; A61K 31/427; A61P 25/00

(52) U.S. Cl. 514/369; 514/517; 514/374; 514/570; 514/450

(57) ABSTRACT

Embodiments of the invention relate to the treatment of neuropathic pain in mammals. Embodiments of the invention include methods for treating neuropathic pain as well as methods for preparing medicaments used in the treatment of mammalian pain. Preferably, methods of the invention comprise the use of PPARgamma agonists for the treatment of mammalian pain.

### CROSS REFERENCE TO RELATED APPLICATIONS

This application claims the benefit of U.S. Provisional Application 60/864,095, filed Nov. 2, 2006, which is hereby incorporated herein by reference in its entirety.

### FIELD

Embodiments of the invention relate to the treatment of pain, including neuropathic pain, in mammals.

### BACKGROUND

### Neuropathic Pain

Pain is the most common symptom for which patients seek medical help, and can be classified as either acute or chronic. Acute pain is precipitated by immediate tissue injury (e.g., a burn or a cut), and is usually self-limited. This form of pain is a natural defense mechanism in response to immediate tissue injury, preventing further use of the injured body part, and withdrawal from the painful stimulus. It is amenable to traditional pain therapeutics, including non-steroidal anti-inflammatory drugs (NSAIDs) and opioids. In contrast, chronic pain is present for an extended period, e.g., for 3 or more months, persisting after an injury has resolved, and can lead to significant changes in a patient's life (e.g., functional ability and quality of life) (Foley, Pain, In: Cecil Textbook of Medicine, pp. 100-107, Bennett and Plum eds., 20th ed., 1996).

Chronic debilitating pain represents a significant medical dilemma. In the United States, about 40 million people suffer from chronic recurrent headaches; 35 million people suffer from persistent back pain; 20 million people suffer from osteoarthritis; 2.1 million people suffer from rheumatoid arthritis; and 5 million people suffer from cancer-related pain (Brower, Nature Biotechnology 2000; 18:387-191). Cancer-related pain results from both inflammation and nerve damage. In addition, analgesics are often associated with debilitating side effects such as abuse potential nausea, dizziness, constipation, respiratory depression and cognitive dysfunction (Brower, Nature Biotechnology 22000; 18:387-391). Pain can be classified as either “nociceptive” or “neuropathic”, as defined below.

“Nociceptive pain” results from activation of pain sensitive nerve fibers, either somatic or visceral. Nociceptive pain is generally a response to direct tissue damage. The term “neuropathic pain” refers to pain that is due to injury or disease of the central or peripheral nervous system. In contrast to the immediate pain caused by tissue injury, neuropathic pain can develop days or months after a traumatic injury. Furthermore, while pain caused by tissue injury is usually limited in duration to the period of tissue repair, neuropathic pain frequently is long lasting or chronic. Moreover, neuropathic pain can occur spontaneously or as a result of stimulation that normally is not painful. Unfortunately, neuropathic pain is often resistant to available drug therapies; a hallmark of neuropathic pain is its intractability. Typical non-steroidal anti-inflammatory drugs (NSAIDs) such as aspirin, indomethecin, and ibuprofen do not relieve neuropathic pain. The neuropathic pain observed in animal models predictive of human clinical outcome does not respond to NSAIDs. Treatments for neuropathic pain include opioids, anti-epileptics, NMDA antagonists, topical Lidocaine, and tricyclic anti-depressants. Current therapies may have serious side effects such as abuse potential, cognitive changes, sedation, and nausea. Many patients suffering from neuropathic pain have limited tolerance of such side effects.

### PPAR Gamma Signaling Pathway and Modulators Thereof

Background information related to PPAR may be found in, WO 2001028540, Polymorphisms in the human insulin receptor gene as drug targets for therapy of cephalic pain, Purvis, Ian James; McCarthy, Linda Catherine; WO 2001028539, Polymorphisms in the human insulin receptor gene as drug targets for therapy of cephalic pain, Purvis, Ian James; McCarthy, Linda Catherine; US 20030046719, Proliferator-Activated Receptor Disruption, compositions and Methods Relating Thereto, Keith D. Allen, Catherine Guenther, Russell Phillips, Mar. 6, 2003; US20030212138, Combinations of Peroxisome Proliferator-Activated Receptor-Alpha Agonists and Cyclooxygenase-2 Selective Inhibitors and Therapeutic Uses Therefor, Mark G. Obukowicz, Nov. 13, 2003; US20060116416, PPAR Active Compounds, Jack Lin, Dean R. Artis, Prabha N Ibrahim, Chao Zhang, Rebecca Zuckerman, Ryan Bremer, Shenghua Shi, Byunghun Lee, Jun. 1, 2006; US20060135540, PPAR Active Compounds, Jack Lin Prabha N. Ibrahim, Dean R. Artis, Chao Zhang, Weiru Wang, Shenghua Shi, Jun. 22, 2006; WO2002100351, A Method for Treating Inflammatory Diseases By Administering a PPAR-Delta Agonist, Michael J. Forrest, Joel, P. Berger, David E. Moller, Samuel Wright, Dec. 19, 2002; WO2005115370, Compositions and Methods for Treating Non-Inflammatory Pain Using PPAR Alpha Agonists, Daniele Piomelli, Jesse Loverme, Dec. 8, 2005; WO2006045581, The Use of 1,2,4-Thiadiazolidine-3,5-Diones as PPAR Activators, Ana Martinez Gil, Mercedes Alonso Cascon, Maria Luisa Navarro Rico, Miguel Medina Padilla, Susana Morales Alcelay, Ana Perez Castillo, Rosario De Luna Medina, May 4, 2006; WO2006078605, Methods of Use of Dual PPAR Agonist Compounds and Drug Delivery Devices Containing Such Compounds, David Saul Cohen, Jul. 27, 2006; WO2006085686, Remedy for Neurogenic Pain, Aug. 18, 2006.

The peroxisome proliferator-activated receptors (PPARs; α β/δ and γ) are a subfamily of ligand-inducible nuclear hormone transcription factors with roles in a range of physiological processes and disease states. PPARγ is expressed in tissues important for insulin action such as adipose tissue, skeletal muscle and liver. In the treatment of diabetes, activation of PPARγ improves glycemic control by improving insulin sensitivity, via activation of genes involved in the control of glucose production, transport and utilization.

Alternatively, PPARα is localized in tissues of the heart, liver and muscle, where it plays an important role in lipid metabolism by controlling genes relating to cellular free fatty acid metabolism and cholesterol trafficking PPARα activation decreases serum triglycerides (TGs) and increases levels of serum high-density lipoprotein (HDL)-cholesterol [622625]. Hypertriglyceridemia and low serum HDL-cholesterol are characteristic of both diabetic dyslipidemia and insulin resistance syndrome.

Multiple literature references associate PPARα and γ signaling with inflammation, and by inference, inflammatory pain. For example, Burstein et al. (Life Sciences. 2004. pg. 751513-1522) postulates that the mechanism of action of ajulemic acid, a cannabinoid produces analgesia without a “high” acts through the PPARγ receptor. However, inflammatory pain is distinct from neuropathic pain. And in fact, ajulemic acid demonstrates potent anti-inflammatory activity (Zurier et al., 1998) and, only demonstrates analgesic effects on a variety of inflammatory pain models which are poor predictors of human outcome including the formalin assay, the PPQ writhing test, the hot plate assay and the tail clip assay.

Due to its effect on insulin resistance and glucose metabolism, multiple literature references associate PPAR α and γ signaling with diabetes, and by inference pain associated with diabetic neuropathy.

One meeting abstract in May of 2006 was presented at the American Pain Society meeting in San Antonio, Tex. (May 3-6, 2006). The abstract describes the intrathecal administration of putative PPARγ agonists 15dPGJ2 and rosiglitazone to rats undergoing the partial sciatic nerve lesion (Seltzer model) of neuropathic pain. While a reduction in pain behavior was observed, the authors indicate that it is not obvious the effect was a PPARγ receptor-mediated effect. The mode of action of thiazolidinediones (TZDs) including rosiglitazone is uncertain, because TZDs were originally developed through the screening of clofibric acid analogues for antilipidaemic and antihyperglycaemic potential, without any knowledge of their molecular target (Kawamatsu Y, Saraie T, Imamiya E, Nishikawa K, Hamuro Y. Studies on antihyperlipidemic agents. I. Synthesis and hypolipidemic activities of phenoxyphenyl alkanoic acid derivatives. Arzneimittelforschung 1980; 30: 454-459.) In fact, rosiglitazone is known to demonstrate activity at PPARα and γ, and one patent publication (in more detail below) proposes the antagonism (as opposed to agonism) of PPARγ for use in the treatment of neuropathic pain.

In patent application WO2005115370, Compounds and Methods for Treating Non-inflammatory Pain using PPARα Antagonists, Piomelli et al. state: “Compounds and methods for treating noninflammatory pain, including but not limited to, neuropathic pain by using peroxisome proliferator activated receptor α agonists to treat a subject having such pain are described. The agonists may be used with additional therapeutic agents such as an inhibitor of fatty acid amide hydrolase or a cannabinoid CB1 or CB2 cannabinoid receptor agonist.”

In patent application WO2006085686, Remedy for Neurogenic Pain, Tanabe & Tsutomu, Tokyo Medical & Dental University state: “ . . . it is intended to provide a remedy for neurogenic pain which contains, as the active ingredient, a PPARγ antagonist (such as 2-chloro-5-nitro-N-phenylbenzamide) . . . a medicinal composition for treating neurogenic pain which contains, as the active ingredient, a PPAR antagonist . . . .” Tanabe and Tsutomu demonstrate that GW9662, a PPARγ antagonist demonstrates activity in a neurogenic pain model. Results are shown in FIG. 1.

In a study of thiazolidinediones (TZDs) published after the priority date of the instant patent application, Park et al. (December 2006. J Pharmacol Exp Ther.) examine the effects of the compounds on spinal cord injury (SCI). Thiazolidinediones (TZDs) block inflammation and induce neuroprotection after ischemia. The study tested TZD effects on spinal cord injury (SCI) on lesion size, motor neuron loss, myelin loss, astrogliosis, and microglia activation. TZDs are known to have anti-inflammatory effects. The investigators induced spinal cord injury and immediately treat with TZDs to reduce inflammation. By measuring markers of injury (lesion size, motor neuron loss, myelin loss, astrogliosis, and microglia activation) and performing a time course of TZD administration from 5 m to 2 d after SCI, they conclude that the treatment was effective for reducing inflammatory injury only if the injection was given by 2 h after the injury by SCI. They also specifically measured TZD effects on pro-inflammatory transcription factors and concluded that TZD treatment following SCI mechanistically prevented such markers of inflammation. When inflammation reduction by TZDs after SCI was most potent (2 h after injury), not surprisingly they observed a reduction of thermal hyperalgia (neuropathic pain) at t=28 d following TZD treatment. In the SCI model, it takes 2 to 5 weeks after SCI to develop chronic neuropathic pain. A hallmark of neuropathic pain is its intractability to amelioration by NSAIDs (non-steroidal anti-inflammation drugs). Therefore in the animal models, including SCI, neuropathic pain is defined as the non-inflammatory component remaining after sufficient time (2 to 5 weeks in SCI) has passed for the inflammation associated with the original injury to resolve itself. It is not obvious in Park et al. whether the apparent analgesic outcome is due to amelioration of neuropathic pain or due to TZD reduction of the initial inflammatory injury, since reduced injury would reduce the severity of subsequent neuropathic pain. In fact, given the early dosing, 2 h immediately after injury, such administration of a TZD as therapy for anticipated neuropathic pain by reduction of inflammation at injury, comprises a poor therapeutic strategy. Patients suffering from neuropathic pain seek therapy long after the initial injury, usually when the original source of nerve injury is unknown. Park et al does not teach the use of TZDs to treat neuropathic pain because the administration was prior to the establishment of neuropathic pain at 2 to 5 weeks after injury in the model they used. In addition, contradicting Tanabe and Tsutomu, Park et al. demonstrate that the same PPARγ antagonist, GW9662, reverses apparent amelioration of subsequent neurogenic pain when co-administered with PPARγ agonist pioglitazone immediately following injury. Results are illustrated in FIG. 2.

In another publication after the priority date of the instant patent application, Churi et al. (February 2007. Meeting report: American Academy of Pain Medicine) further investigate the specific mechanism of action for rosiglitazone and 15dPGJ2. Intrathecal administration of rosiglitazone and 15dPGJ2 in spared nerve injury (SNI) 7 days post-SNI, 15dPGJ2 dose-dependently reduced mechanical allodynia (von Frey). Rosiglitazone reduced mechanical and cold allodynia (acetone). Observed effects were reversed by PPARγ antagonist BADGE. The findings confirm the inventors innovation that “ . . . activation of spinal PPARγ reverses mechanical allodynia. Our results suggest that new or currently available drugs targeted at spinal PPARγ may yield important therapeutic effects for the treatment of neuropathic pain.” Taken all together, the prior art showed that PPAR antagonist GW9662 decreased neurogenic pain and putative PPARγ agonist rosiglitazone also decreased neurogenic pain. Churi et al. conclude that all drugs targeted at spinal PPARγ may yield important therapeutic effects for the treatment of neuropathic pain based on reversal of the rosiglitazone effect in SNI by putative PPARγ antagonist BADGE. However, Bishop-Bailey et al. (Bisphenol A diglycidyl ether (BADGE) is a PPARγ agonist in an ECV304 cell line. 2001. British Journal of Pharmacology 131:651-654) conclude “ . . . the only compound to be previously described as a pure PPARγ antagonist, has PPARγ agonist activity. These results indicate that care must be taken when using BADGE as a pharmacological tool to look at the role of PPARγ.”Therefore, the prior art does not teach or make obvious whether PPARγ ligands, as a class, or any particular PPARγ ligand will or will not reduce neurogenic pain.

Tesaglitazar. PPARγ Agonist/PPARα Agonist

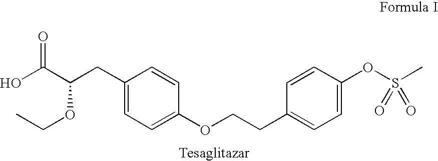

Tesaglitazar is disclosed and discussed in U.S. Pat. No. 6,258,850; and U.S. patent application publication 2004/0152771; AstraZeneca AB [AstraZeneca plc] (Patent Assignee/Owner), A pharmaceutical combination comprising either (S)-2-ethoxy-3-[4-(2-{4-methanesulfonyl oxyphenyl}ethoxy)phenyl]propanoic acid or 3-{4-[2-(4-tert-butoxy carbonyl aminophenyl)ethoxy)phenyl}-(S)-2-ethoxy propanoic acid and a biguanide drug, WO-02096402 5 Dec. 2002 (1 Jun. 2001); AstraZeneca AB [AstraZeneca plc] (Patent Assignee/Owner), Process for the preparation of 3-aryl-2-hydroxypropionic acid derivative, WO-02096865 5 Dec. 2002 (1 Jun. 2001); AstraZeneca AB [AstraZeneca plc] (Patent Assignee/Owner), A pharmaceutical combination comprising either (S)-2-ethoxy-3[4-(2-{4-methanesulfonyl oxyphenyl}ethoxy)phenyl]propanoic acid or 3-{4-[2-(4-tert-butoxy carbonylaminophenyl)ethoxy]phenyl}-(S)-2-ethoxy propanoic acid and insulin, WO-02096453 5 Dec. 2002 (1 Jun. 2001); AstraZeneca AB [AstraZeneca plc] (Patent Assignee/Owner), A pharmaceutical combination comprising either (S)-2-ethoxy-3-[4-(2-{4-methane sulfonyl oxyphenyl}ethoxy)phenyl]propanoic acid or 3-{4-[2-(4-tert-butoxy carbonyl aminophenyl)ethoxy]phenyl}-(S)-2-ethoxy propanoic acid and a sulfonylurea, WO-02100413 19 Dec. 2002 (1 Jun. 2001); AstraZeneca AB [AstraZeneca plc] (Patent Assignee/Owner), Comminuted form of (S)-2-ethoxy-3-[4-(2-{4-methanesulfonyloxyphenyl}ethoxy)phenyl]propanoic acid, WO-00140169 07 Jun. 2001 (3 Dec. 1999); AstraZeneca AB [AstraZeneca plc] (Patent Assignee/Owner), Crystalline form of (S)-2 ethoxy-3-[4-(2-{4-methanesulfonyloxyphenyl}ethoxy)phenyl]propanoic acid, WO-00140171 7Jun. 2001 (3 Dec. 1999); Original Assignee(s), New Process, WO-00140159 07Jun. 2001 (3 Dec. 1999); Astra AB [AstraZeneca plc] (Patent Assignee/Owner), New 3-aryl-2-hydroxypropionic acid derivative (I), WO-09962872 09 Dec. 99 (4 Jun. 98).

AstraZeneca has discontinued development of tesaglitazar (Galida), an oral dual PPAR α/γ agonist, which was being investigated for the potential treatment of type II diabetes and lipid disorders. Development was discontinued following a review of data from four phase III trials and one phase II trial which found the tesaglitazar risk/benefit profile was unlikely to offer patients a significant advantage over existing therapies.

Preclinical Data

The PPAR agonist activity of tesaglitazar was demonstrated by studies of the ligand binding and activation of the receptor. The binding of tesaglitazar to PPARalpha and γ led to the recruitment of steroid receptor co-activator (SRC)-1 with comparable ED50 values of 1.2 and 1.3 microM, respectively, and stabilized the AF2 helices of the LBDs.

The activation of the PPAR subtypes was compared in reporter gene assays in U2 OS cells treated with agonists. For tesaglitazar, respective EC50 values for mouse (m)PPARgamma, mPPARalpha and hPPARalpha were 0.25, 32 and 1.7 microM. At the same respective receptors, EC₅₀ values for bezafibrate were 23, 24 and 17 microM. This was compared with EC50 values of 0.05 microM for rosaglitazone at PPARgamma, and 0.20 microM for WY-14643 at mPPARalpha; no activity was determined for these compounds at the other PPARs.

In human HepG2 cells exposed to tesaglitazar or bezafibrate (8 and 71 microM, respectively), the induction of a known PPARalpha target protein was reported to be qualitatively similar (˜3- to 4-fold as determined by proteomic methods); thus, tesaglitazar was approximately 10-fold more potent than bezafibrate.

In an examination of the potential role of tesaglitazar in reverse cholesterol transport, the drug (5 microM) improved the capacity of human macrophages to export cholesterol to HDL. When cells were exposed to high concentrations of fatty acids and TGs, cholesterol efflux was reduced to 80.8% of the level observed in control cells; however, treatment with tesaglitazar increased efflux to 156% of the control level. This result indicated that tesaglitazar might contribute to an anti-atherogenic effect.

The dyslipidemia resulting from diabetes and insulin resistance exposes arterial cells to elevated levels of low-density lipoproteins (LDLs), which are thought to accumulate in the arterial intima, entrapped by proteoglycans. A study examined the ability of tesaglitazar to block the changes induced by the fatty acid linoleate to glycosaminoglycans (GAGs) isolated from arterial smooth muscle cells (SMCs). Tesaglitazar (5 or 10 microM) abolished linoleate-induced production of LDL-binding GAGs and additionally decreased the affinity of human LDL for unbound GAGs by 3- to 4-fold.

The upregulation of cytochrome P450 (CYP)4A is a known consequence of PPARalpha activation, and was studied in a B6C3F1 lean mouse model. Tesaglitazar (0.13 microg/kg) caused the upregulation of CYP4A by 15-fold relative to controls, whereas WY-14643 caused a 22-fold upregulation. Rosiglitazone lacked this effect, predictably, due to its PPARgamma specificity.

The efficacy of tesaglitazar in restoring insulin sensitivity has been evaluated in a number of animal models. In several similar studies in obese insulin-resistant Zucker rats, tesaglitazar (3 micromol/kg/day), administered orally for 3 or 4 weeks, consistently improved or normalized measures of whole-body insulin sensitivity to levels approaching those of lean rats, in both basal and hyperinsulinemic states, compared with untreated obese animals. In one study, tesaglitazar produced improvements in several measures of insulin sensitivity (expressed as percentage normalization of mean values toward lean control values in treated versus untreated obese animals) during basal and hyperinsulinemic euglycemic clamp conditions. In basal and clamp conditions, respectively, normalizations were observed in levels of total insulin (76 and 82%), C-peptide (61 and 63%), TGs (67 and 72%) and free fatty acids (15 and 70%). During clamp conditions, the rate of glucose infusion increased by 7.6-fold in tesaglitazar-treated rats, compared with untreated rats, a 13% higher infusion rate than required in lean controls. In a similar study, tesaglitazar treatment normalized postprandial excursions in plasma glucose (80%), insulin (85%), TGs (92%) and free fatty acids (75%), following administration of a glucose/TG (1.7/2.0 g/kg) test meal. In fasted, obese Zucker rats and obese controls, tesaglitazar reduced the rate of hepatic TG production by 47%, increased plasma TG clearance by 490% and reduced very (V) LDL apolipoprotein (apo)CIII content.

In the human apoB/cholesteryl ester transfer protein (CETP) double-transgenic mouse, a model that exhibits a ‘humanized’ lipoprotein profile and develops insulin resistance when administered a diet high in fat and sucrose, tesaglitazar (1 microM/kg/day for 2 weeks) reduced levels of plasma TGs and apoB100, and also reduced plasma CETP activity, to the same extent as animals treated with the selective PPARalpha agonist WY-14643. In comparison, rosiglitazone had no such effect.

Similar findings were reported in the non-genetic, high-fat-fed Wistar rat model. Tesaglitazar (1 micromol/kg/day for 1 week) reduced basal plasma insulin levels (36%), increased glucose infusion rate during clamping (29%) and reduced basal TG levels.

In obese, diabetic ob/ob mice, treatment with tesaglitazar (1 micromol/kg/day) for 1 week normalized hyperglycemia and reduced insulin levels, relative to untreated obese animals, resulting in the reduction of TG levels to below those of lean mice. In terms of the dose required for a 25% reduction in average fasting plasma glucose, insulin and serum TG levels, tesaglitazar was 7-fold more potent than rosiglitazone and 250-fold more potent than pioglitazone.

Several animal studies assessed the effect of tesaglitazar on the metabolic flexibility (specifically the capacity of skeletal muscle to switch from utilizing fatty acids to utilizing glucose) that is impaired in insulin resistance. In high-fat-fed Wistar rats, tesaglitazar (1 micromol/kg/day for 3 weeks) increased the uptake of non-esterified fatty acids in white adipose tissue under basal conditions (52%), and modestly increased fatty acid clearance in hyperinsulinemic euglycemic clamp conditions; no effect on clearance was reported in red gastrocnemius muscle in either condition. The utilization of fatty acids was modestly increased in the liver and muscle. This result differs from the findings of a study in obese Zucker rats, in which, under insulin level-clamped conditions, animals treated with tesaglitazar (3 micromol/kg/day for 3 weeks) showed reduced free fatty acid utilization in adipose tissue, skeletal muscle, liver and heart. The apparent discrepancies between these studies may have resulted from differences in the animal models, tracer compounds, and perhaps also the drug dosage. In the latter study, tesaglitazar increased glucose utilization in muscle and fat, leading to the normalization of glycogen stores in both tissues.

Studies were also conduced in apoE*3 Leiden transgenic mice, a dominant-negative apoE mutant model that is thought to better mimic the lipoprotein profile of humans. In high cholesterol-fed mice, tesaglitazar (0.5 microg/kg of diet) reduced plasma cholesterol levels by approximately 21.5% in animals fed either low- or high-fat (to induce insulin resistance) diets, relative to controls fed in the same manner, and reduced TG levels by approximately 40%. Low- and high-fat fed animals were treated for 16 and 28 weeks, respectively; the greater period of treatment for the latter animal is due to reports that lesions take longer to develop with the latter diet. Tesaglitazar treatment also reduced the cross-sectional area of atheromic lesions in the aortic root by 65 and 92% in low- and high-fat fed animals, respectively. This reduction was greater in mice receiving tesaglitazar than in animals in which the level of plasma cholesterol had been titrated down to levels equivalent to the reduction by tesaglitazar, suggesting an effect greater than that attributable to cholesterol-lowering effects.

Metabolism and Pharmacokinetics

In vitro, the uridine diphosphate glucuronyl transferase (UGT) isoforms UTG1A3 and UGTB7 were shown to be the key glucuronating isoenzymes for tesaglitazar in experiments using human liver microsomes. This finding is consistent with data from studies in rats, dogs and healthy humans that identified tesaglitazar acylglucuronide to be the main metabolite. Tesaglitazar and its acylglucuronide metabolite were not substrates of P-glycoprotein and, in Caco2 and MDCK-MDR1 cell monolayer models, were apparently transported by multidrug resistance protein 2.

The effect of tesaglitazar on the activity of important CYP drug metabolizing enzymes was assessed using seven recombinant human CYPs (CYP1A2, CYP2A6, CYP2C8, CYP2C9, CYP2C19, CYP2D6 and CYP3A4 expressed in yeast), which together account for the metabolism of 90% of currently used drugs. Tesaglitazar had no effect on any of these CYPs when tested at concentrations that extend greatly beyond anticipated in vivo levels (from 0.09 to 200 microM).

Tesaglitazar has been assayed in plasma using techniques such as liquid chromatography-mass spectrometry with solid- or liquid-phase extraction. In one study, the pharmacokinetics of [¹⁴C]tesaglitazar were studied in eight healthy men in an open-label crossover phase II clinical trial. Each volunteer received a single 1-mg dose of labeled tesaglitazar by oral or intravenous routes, followed by a washout period and then administration of the alternative formulation. The drug was rapidly absorbed after oral dosing, with a median Tmax value of ˜0.5 h and a Cmax value of 0.61 microM. Oral bioavailability was ˜100%, implying limited first-pass metabolism. Most of the drug was eliminated in the urine as the acylglucuronide metabolite. The drug was highly protein-bound (99.9%), with a low clearance (Cl=0.16 l/h), a small volume of distribution at steady state (Vdss=9.1 l) and an elimination half-life of ˜45 h. Pharmacokinetic parameters were highly similar following intravenous and oral dosing (respective AUC values of 16.2 and 16.1 micromolxh/l) as a result of the high oral bioavailability [609697]. A single-dose clinical trial in 20 healthy males administered 1 mg of tesaglitazar with or without food demonstrated that oral bioavailability was not influenced by food, although Tmax was slightly extended in the fed state (2.5 h) versus the fasted state (1 h).

These findings were confirmed in a 12-week clinical trial in non-diabetic patients (n=240) of both sexes with manifestation of insulin resistance; following once-daily oral administration of tesaglitazar (0.1 to 1 mg), the pharmacokinetic profile fitted a one-compartment model with first-order absorption and elimination.

No pharmacokinetic interactions were found between tesaglitazar and either of the commonly used antidiabetic agents glibenclamide or metformin. In the first of two crossover trials in healthy males, 16 volunteers were randomized to receive daily oral doses of 8 mg of tesaglitazar, 3.5 mg of glibenclamide or the combination, for 1 week each, separated by a 3-week washout period. The AUC and Cmax values of the respective drugs were similar whether given alone or in combination. An identical design was used to confirm the lack of interaction between tesaglitazar and metformin. In this second trial, 14 males received oral doses of 500 mg of metformin twice-daily, 3 mg of tesaglitazar once-daily, or the combination, and no significant interactions were observed.

Clinical Development

The efficacy of tesaglitazar has been confirmed in several phase II clinical trials. In the randomized, double-blind, dose-ranging GLAD (glucose and lipid assessment in diabetes) study, 488 type 2 diabetic patients received once-daily oral doses of tesaglitazar (0.1, 0.5, 1.0, 2.0 or 3.0 mg), open-label pioglitazone (45 mg) or placebo, for 12 weeks. Results were provided in the form of placebo-corrected changes from baseline. Tesaglitazar dose-dependently decreased mean levels of fasting plasma glucose (from 170.4 mg/dl at baseline) by 8.9, 30.3, 41.1, 55.0 and 60.9 mg/dl in the ascending 0.1- to 3.0-mg dose groups, respectively, compared with a reduction of 38.5 mg/dl in the pioglitazone group. Significant reductions were achieved at the 0.5-mg dose of tesaglitazar and above. Improvements in the serum lipid profile were also reported across the dose range. For tesaglitazar, the maximum reduction in serum TGs (41.0%) and free fatty acids (36.7%) occurred at the 2-mg dose, and the greatest reduction of LDL-cholesterol (17.3%) and VLDL-cholesterol (52.5%) occurred at 3 mg. The greatest increase in serum HDL-cholesterol (15.0%) was observed at 1 mg. In comparison, pioglitazone (45 mg) produced less pronounced changes, with reductions in levels of TGs (7.6%), free fatty acids (21.8%) and LDL-cholesterol (4.4%), and increases in HDL-cholesterol (5.8%). Unlike with any dose of tesaglitazar, the level of VLDL-cholesterol increased (6.4%) with pioglitazone.

In another randomized, double-blind, placebo-controlled trial, 390 non-diabetic but insulin-resistant patients received either once-daily oral doses of tesaglitazar (0.1, 0.25, 0.5 or 1 mg) or placebo for 12 weeks. This study (SH-SBT-0001) was part of the SIR (study in insulin resistance) trials.

At baseline, all patients had an abnormal waist to hip ratio (men >0.90, women >0.85) and a serum TG level >/=150 mg/dl (>/=1.7 mmol/l) [608130]. Following the administration of 1 mg of tesaglitazar, placebo-corrected results showed significant mean reductions in fasting levels of serum TGs (37%), non-HDL-cholesterol (15%), non-esterified fatty acids (40%), insulin (35%) and plasma glucose concentration (0.47 mmol/l); serum HDL-cholesterol, on the other hand, increased (16%). In the same sample, tesaglitazar exerted a dose-dependent beneficial effect on dysregulated apolipoprotein abnormalities. The 1-mg dose significantly reduced levels of ApoB (12%), ApoCIII (25%), and reduced the ApoB/ApoA-1 ratio (16%; all p<0.0001), while the level of ApoA-1 was increased (4%; p<0.05). Postprandial lipid handling was examined in a subpopulation of 222 patients. Following a lipid-rich meal, 1 mg of tesaglitazar significantly and dose-dependently reduced the AUC values for serum TGs (41%), plasma free fatty acids (29%), serum glycerol (34%), plasma insulin (31%) and plasma glucose level at 2 h (27%). After 12 weeks of receiving 1 mg of tesaglitazar, all patients had normal glucose tolerance, compared with 85% at baseline. Tesaglitazar was also effective at reducing the prevalence of metabolic syndrome, assessed according to the National Cholesterol Education Program (NCEP) ATPIII criteria. The 0.5- and 1.0-mg doses reduced the prevalence by 49 and 45%, respectively, compared with only a 6% reduction in the control group. The prevalence of impaired fasting glucose fell by 23 and 59% (in 0.5- and 1.0-mg dose groups, respectively) compared with an increase of 22% in the placebo group.

In a small-scale pharmacokinetic study of tesaglitazar (1 mg po or iv) in healthy volunteers, no serious adverse events were reported and there were no clinically significant changes in electrocardiogram, blood pressure, heart rate or routine laboratory variables. Similarly, no adverse effects or pharmacokinetic interactions were observed between tesaglitazar and glibenclamide or metformin.

In a dose-ranging phase II clinical trial in non-diabetic, insulin-resistant patients, no association was found between the frequency of adverse events and the tesaglitazar dose. However, a dose-dependent reduction in mean hemoglobin level was recorded, ranging from 0.16 to 0.55 mmol/l in the 0.1- and 1-mg does groups, respectively. No cases of heart failure were observed. There was also a reversible dose-related increase in serum creatinine (from a mean of 1 to 8 micromol/l in the 0.1- and 1.0-mg tesaglitazar groups, respectively) that occurred during the first month of treatment, which then stabilized. Several cases of edema were reported but these were not clearly related to use of active medication or dose. Adverse events occurred in 65, 51, 67 and 60% of patients in the 0.1-, 0.25-, 0.5- and 1.0-mg doses, respectively, compared with 55% in the placebo group.

The effect of tesaglitazar on body weight is difficult to assess from the available clinical data because of the limited duration of drug exposure (up to 12 weeks only). Nevertheless, there was a small but statistically significant increase in weight of approximately 1 kg in the highest dose groups (0.5 and 1 mg) in the 12-week study of insulin-resistant patients.

In a phase II clinical trial of diabetic patients allocated tesaglitazar, pioglitazone or placebo, rates of edema did not differ significantly between treatment groups (4.2 to 6.8% for tesaglitazar-, 4.2% for pioglitazone- and 2.9% for placebo-treated patients). However, patient numbers were too small for a valid comparison to be made.

Edaglitazone. PPARγ Agonist

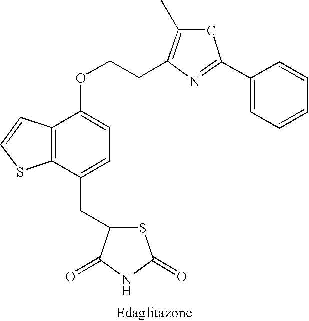

Edaglitazone is disclosed and discussed in the following references: F Hoffmann-La Roche Ltd [Roche Holding AG] (Patent Assignee/Owner), Process for the preparation of insulin sensitizer and intermediate compound thereof, WO-2005000844 6 Jan. 2005 (26 Jun. 2003); Hoffmann-La Roche AG [Roche Holding AG] (Patent Assignee/Owner), Thiazolidinediones alone or in combination with other therapeutic agents for inhibiting or reducing tumor growth, WO-02080913 17 Oct. 2002 (6 Apr. 2001); Boehringer Mannheim GmbH [Roche Holding AG] (Patent Assignee/Owner), Improved method for producing thiazolidinediones, and new thiazolidinediones, WO-09842704 1 Oct. 1998 (20 Mar. 1997); Boehringer Mannheim GmbH [Roche Holding AG] (Patent Assignee/Owner), New thiazolidindiones and drugs containing them, WO-09427995 08 Dec. 1994 (25 May 1993); U.S. Pat. No. 5,599,826; and U.S. Pat. No. 7,259,176. These references also disclose a genus of PPARγ agonists of formula:

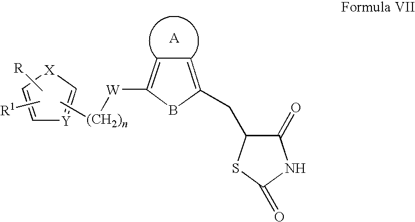

wherein:

A is a carbocyclic ring with 5 or 6 carbon atoms or a heterocyclic ring with a maximum of 4 heteroatoms in which the heteroatoms can be the same or different and denote oxygen, nitrogen, or sulfur and the heterocycles can if desired, carry an oxygen atom on one or several nitrogen atoms;

B is —CH═CH—, —N═CH—, —CH═N—, O, or S;

W is CH2, OCH(OH), CO or —CH═CH—;

X is S, O, or NR2 in which the residue R2 is hydrogen or C1-6 alkyl;

Y is CH or N;

R is naphthyl, pyridyl, furyl, thienyl, or phenyl which if desired is mono- or disubstituted with C1-3 alkyl, CF3, C1-3 alkoxy, F, Cl, or Br;

R1 is hydrogen or C₁₋₆ alkyl;

n is 1 to 3; and

tautomers, enantiomers, diasteromers, and pharmaceutically acceptable salts, hydrates, solvates, prodrugs, and polymorphs thereof.

Roche (formerly Boehringer Mannheim) and its Japanese subsidiary Chugai were developing edaglitazone, an orally administered PPARγ agonist, for the potential treatment of type 2 diabetes. Phase II trials were complete by May 2004, and at that time, plans were underway to initiate phase III trials. However, by July 2004, following new guidance by the FDA on the class of drugs to which edaglitazone belongs, Roche had revised its phase III development plans to wait for the results of ongoing long-term toxicity studies. In April 2006, Roche reported that the edaglitazone program had been discontinued.

Preclinical Data

Doses of 1, 10, 25, 50 and 100 mg/kg of edaglitazone for 12 days in db/db mice led to dose-dependent reductions in non-starved blood glucose levels. The 25-mg dose led to a 52% lowering, while chronic treatment with 100 mg/kg did not result in hypoglycemia. A dose of 1 mg/kg edaglitazone caused a 14% reduction in serum triglyceride levels, while the 10-mg·kg dose produced a 70% reduction. Free fatty acid levels were lowered by 16 and 70% following 1- and 10-mg/kg doses, respectively. In ob/ob mice administered between 0.25 and 10 mg/kg doses of edaglitazone for 12 days, reductions in non-starved blood glucose concentration of 29% were seen. Higher doses produced a 45% reduction. Serum insulin levels were dose-dependently decreased at all doses, while after 14 days of treatment with 1 mg/kg, the AUC(glucose) was significantly reduced by 44%.

Edaglitazone induced distinct insulin-sensitization but did not affect basal rates of glycogen synthesis. It also increased the rate of glucose oxidation in both the presence and absence of insulin

Clinical Data

In a single-ascending-dose study, 56 healthy males received 1, 3, 10, 20, 40, 80, and 160 mg edaglitazone (n=6 per group). Edaglitazone was very well tolerated and no edema or hypoglycemia was reported in any patient, and AUC and Cmax increased dose-proportionally over the entire dose range. Absorption of edaglitazone was relatively fast, and plasma peak levels were achieved within 2 to 4 h after dosing. In a multiple-ascending-dose study, edaglitazone did not accumulate following once-daily dosing regimen over 6 weeks.

Farglitazar. PPARγ Agonist; Retinoid X Receptor Modulator

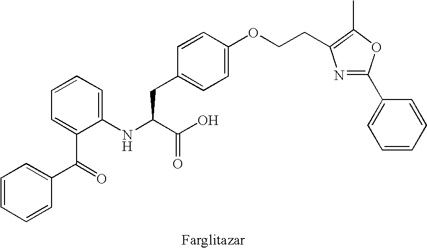

Farglitazar is disclosed and discussed in SmithKline Beecham Corp [GlaxoSmithKline plc] (Patent Assignee/Owner), Novel therapeutic method and compositions for topical administration WO-2004073627 02 Sep. 2004 (17 Feb. 2003); SmithKline Beecham Corp [GlaxoSmithKline plc] (Patent Assignee/Owner) Dosing regimen for PPARγ activators WO-03055485 10 Jul. 2003 (21 Dec. 2001); Glaxo Wellcome plc [GlaxoSmithKline plc] (Patent Assignee/Owner) Diagnostic test WO-00233121 25 Apr. 2002 (19 Oct. 2000); Glaxo Wellcome plc [GlaxoSmithKline plc] (Patent Assignee/Owner) Process for preparing and harvesting crystalline particles WO-00200198 03 Jan. 2002 (29 Jun. 2000); Glaxo Wellcome plc [GlaxoSmithKline plc] (Patent Assignee/Owner) Novel process for preparing and harvesting crystalline particles WO-00200199 03 Jan. 2002 (29 Jun. 2000); Glaxo Wellcome plc [GlaxoSmithKline plc] (Patent Assignee/Owner) Substituted 4-hydroxy-phenylalcanoic acid derivatives with agonist activity to PPARγ. WO-09731907 04 Sep. 1997 (28 Feb. 1996); and U.S. Pat. No. 6,294,580. These references also disclose a genus of PPARγ agonists of formula:

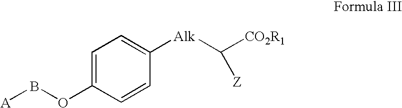

wherein:

A is selected from the group consisting of:
(i) phenyl, wherein the phenyl is optionally substituted by one or more of the following groups: halogen atoms, C₁₋₆ alkyl, C₁₋₃ alkoxy, C₁₋₃ fluoroalkoxy, nitrile, or —NR⁷R⁸ where R⁷ and R⁸ are independently hydrogen or C₁₋₃ alkyl;
(ii) a 5- or 6-membered heterocyclic group containing at least one heteroatom selected from oxygen, nitrogen and sulfur; and
(iii) a fused bicyclic ring

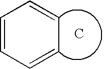

wherein ring C represents a heterocyclic group as defined in point (ii) above, which bicyclic ring is attached to group B via a ring atom of C;

B is selected from the group consisting of:
(iv) C₁₋₆ alkene;
(v) -MC₁₋₆ alkene or C₁₋₆ alkeneMC₁₋₆ alkene, wherein M is O, S, or —NR² wherein R² represents hydrogen or C₁₋₃ alkyl;
(vi) a 5- or 6-membered heterocyclic group containing at least one nitrogen heteroatom and optionally at least one further heteroatom selected from oxygen, nitrogen and sulfur and optionally substituted by C₁₋₃ alkyl; and
(vii) Het-C₁₋₆ alkylene, wherein Het represents a heterocyclic group as defined in point (vi) above;

Alk represents C₁₋₃ alkylene;

R1 represents hydrogen or C₁₋₃ alkyl;

Z is selected from the group consisting of:
(viii) —(C₁₋₃alkylene) phenyl, which phenyl is optionally substituted by one or more halogen atoms; and
(ix) —NR³R⁴, wherein R³ represents hydrogen or C₁₋₃alkyl, and R⁴ represents —Y—(C═O)-T-R⁵, or —Y—(CH(OH))-T-R⁵, wherein:
   (a) Y represents a bond, C₁₋₆ alkylene, C₂₋₆alkenylene, C₄₋₆ cycloalkene or cycloalkenylene, a heterocyclic group as defined in point (vi) above, or phenyl optionally substituted by one or more C₁₋₃ alkyl groups and/or one or more halogen atoms;
   (b) T represents a bond, C₁₋₃ alkyleneoxy, —O— or —N(R⁶)—, wherein R⁶ represents hydrogen or C₁₋₃ alkyl;
   (c) R⁵ represents C₁₋₆ alkyl, C₄₋₆ cycloalkyl or cycloalkenyl, phenyl (optionally substituted by one or more of the following groups; halogen atoms, C₁₋₃ alkyl, C₁₋₃ alkoxy groups, C₀₋₃ alkyleneNR⁹R¹⁰ (where each R⁹ and R¹⁰ is independently hydrogen, C₁₋₃ alkyl, —SO₂C₁₋₃alkyl, or —CO₂C₁₋₃ alkyl, —SO₂NHC₁₋₃alkyl), C₀₋₃ alkyleneCO₂H, C₀₋₃alkyleneCO₂C₁₋₃alkyl, or —OCO₂C(O)NH₂), a 5- or 6-membered heterocyclic group as defined in point (ii) above, a bicyclic fused ring

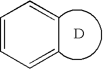

wherein ring D represents a 5- or 6-membered heterocyclic group containing at least one heteroatom selected from oxygen, nitrogen and sulfur and optionally substituted by (═O), which bicyclic ring is attach to T vi a ring atom of ring D: or —C₁₋₆ alkyleneMR¹¹; M is O, S, or NR¹² wherein R¹² and R¹¹ are independently hydrogen or C₁₋₃ alkyl; or a tautomeric form thereof, and/or a pharmaceutically acceptable salt, hydrate, solvate, or polymorph thereof.

The terms C₁₋₃ alkyl or alkylene and C₁₋₆ alkyl or alkylene as used herein respectively contain 1 to 3 or 1 to 6 carbon atoms and appropriately include straight chained and branched alkyl or alkylene groups, typically methyl, methylene, ethyl and ethylene groups, and straight chained and branched propyl, propylene, butyl and butylene groups. The term C₂₋₆ alkenyl or alkenylene as used herein contains 2 to 6 carbon atoms and appropriately includes straight chained and branched alkenyl and alkenylene groups, in particular propenylene or the like;

and pharmaceutically acceptable salts, hydrates, solvates, polymorphs, or prodrugs thereof.

GlaxoSmithKline is developing farglitazar (GW-262570), a peroxisome proliferator-activated receptor (PPAR)-γ agonist and retinoid x receptor modulator, for the potential treatment of hepatic fibrosis, and investigating it for the potential treatment of cardiovascular diseases. In November 2005, farglitazar was in phase II trials for hepatic fibrosis.

The compound was previously under development for type II diabetes, for which it reached phase III trials. However, in October 2001, it was reported that development of the compound for this indication had ceased as it did not meet its target profile. Alternative indications for the compound were being explored at that time, but the company would not disclose these, only revealing that the field would not be obvious to people who had not studied the group of compounds very closely.

Preclinical Data

In Sprague-Dawley rats fed a high-fat/sucrose diet for 4 weeks, treatment with farglitazar 20 mg/kg/day for 2 weeks normalized postprandial serum insulin caused by the high-fat diet, and significantly suppressed serum VEGF (both p<0.01) levels; farglitazar had no effect on serum VEGF of rats on normal diet. In differentiated 3T3 L1 adipocytes, farglitazar modestly increased basal VEGF secretion, but did not affect insulin-increased VEGF secretion. In male Sprague Dawley rats treated with farglitazar 20 mg/kg/day, there was a rapid onset of plasma volume expansion, along with a small but consistent decrease in plasma K+ concentration. Farglitazar treated rats also had a lower plasma levels of aldosterone, although mRNA levels for PPARgamma, the ENaC α subunit, and the glucocorticoid receptor tended to be elevated in kidney medulla by 31, 32, and 13%, respectively, following farglitazar treatment.

Zucker diabetic fatty rats were fed farglitazar (8 mg/kg bid). PPARgamma activation caused an increased capacity of the myocardium to utilize glucose and tighter coupling of oxidative metabolism and contractile performance.

Preclinical studies have demonstrated that rats with early diabetes respond to treatment with farglitazar, however, if treatment is delayed they rapidly progress to levels where control of glycemia is less complete and less durable. Zucker diabetic fatty (ZDF) rats were treated with farglitazar (3 mg/kg/day) starting at age 6 weeks (prior to the onset of diabetes), 8 weeks (diabetic but insulin levels still rising) or 10 weeks (insulin levels falling but still hyperinsulinemic compared with lean litter mates). In ZDF rats treated with farglitazar at 6 weeks, 12 of 13 maintained normal fed glucose levels throughout the 24-week study. Rats treated at 8 weeks had an initial decline in insulin levels which then remained normal throughout the study. At 24 weeks, there was no difference between HbA(1c) levels in rats started on therapy at 6 and 8 weeks compared to controls. However, 0 of 10 rats treated with farglitazar at 10 weeks maintained fed plasma glucose levels, with all gradually increasing to severely diabetic levels.

In male db/db mice, farglitazar (5 mg/kg bid for 14 days) was effective in ameliorating the diabetic phenotype; a significant decrease in non-fasted glucose and insulin suggested an increase in insulin sensitivity in these animals. In ZDF rats, the drug dose-dependently reduced levels of non-esterified fatty acids in this model, as well as lowering levels of triglycerides. These effects were evident within 4 days after the start of dosing.

Data demonstrated that, in the type II db/db male mice model, EC50 values (microM) for PPARalpha and γ agonism were (α/γ): rosiglitazone (qv) 0.1/5; pioglitazone (qv) 1/7; NNC-61-0029 (qv) 0.6/3; JTT-501 (qv) 0.4/2; MCC-555 (qv) 3/0.1; KRP-297 (qv) 0.5/0.4; farglitazar 0.002/0.3; fenoacid ND/32.

Rats receiving 8 mg/kg bid po farglitazar showed PPARγ activation through detection of increased mRNA levels of the target genes FABP3 and aP2. Increased vasodilator NO levels (p<0.05) and fluid retention were observed, while the glomerular filtration rate, effective renal plasma flow and renal filtration fraction were unaffected. It was suggested that increased vasodilator NO levels could contribute to blood pressure lowering brought on by PPARγ activation. In a further study, diabetic rats were administered the drug at 8 mg/kg bid, for 10 days. Compared to controls, systemic vascular pressure and arterial pressure were decreased, while cardiac output was increased. There was no significant change in heart rate. Farglitazar decreased arterial pressure by reducing the total peripheral resistance.

The compound was reported to have cardiovascular effects in conscious rats. A dose of 2 mg/ml at 0.4 ml/h for 2 h bid for 4 days resulted in a fall in the mean arterial blood pressure, tachycardia and marked hindquarters vasodilation in rats.

Preclinical studies demonstrated that farglitazar increased association of the co-activator cAMP response element binding protein and decreased association of the nuclear receptor co-repressor with the retinoid X receptor (RXR). In addition, farglitazar exhibited a 9-fold preference for binding to the RXR-PPARγ complex, compared to the uncomplexed receptor.

Farglitazar is a potent agonist at PPARγ; full activity is seen at 1 nM, although the drug is potent at 0.3 nM. It has a residual effect at PPARα, although three orders of magnitude less than PPARγ, and it is inactive at PPAR δ. The compound has a Ki value against human receptor of <1.2 nM.

Preclinical studies demonstrated that farglitazar increased association of the co-activator cAMP response element binding protein and decreased association of the nuclear receptor co-repressor with the retinoid X receptor (RXR). In addition, farglitazar exhibited a 9-fold preference for binding to the RXR-PPARγ complex, compared to the uncomplexed receptor.

Farglitazar is a potent agonist at PPARγ; full activity is seen at 1 nM, although the drug is potent at 0.3 nM. It has a residual effect at PPARα, although three orders of magnitude less than PPARγ, and it is inactive at PPAR δ. The compound has a Ki value against human receptor of <1.2 nM.

Clinical Data

Farglitazar was well tolerated with no adverse events or significant alterations in laboratory or cardiovascular parameters. The pharmacokinetics of farglitazar were determined in 10 healthy men (23 to 46 years) administered 0.5, 1.5, 5 15 and 40 mg po. Both AUC (44, 111, 355, 963 and 2636 ng·h/ml, respectively) and Cmax (20, 48, 168, 378 and 1173 ng/ml, respectively) were dose-proportional. Half-life (t1/2) values for the respective doses were 3, 3.9, 4.9, 4.9 and 5.3 h. Thus, single oral doses are safe and well tolerated. No pharmacokinetic or pharmacodynamic interaction of farglitazar were detected with warfarin or with digoxin in healthy volunteers.

Farglitazar was studied in a 14-day, randomized, double-blind, placebo-controlled trial in 35 patients with type II diabetes. There were significant reductions in glucose, insulin and triglycerides [422844]. In 376 patients treated for 12 weeks, farglitazar improved metabolic control in type II diabetes mellitus [368659]. In 385 patients treated for 12 weeks, the efficacy of a combination of farglitazar and glibenclamide was superior to glibenclamide alone. Farglitazar (5 and 10 mg/day) significantly lowered blood pressure in hypertensive type II diabetic patients.

It is reported that farglitazar is safe and well tolerated. The most commonly reported adverse events in one clinical trial were headache and gain in bodyweight. The latter effect may be explained by adipocyte differentiation induced by PPARγ activation.

Muraglitazar and Peliglitazar, PPARγ Agonists

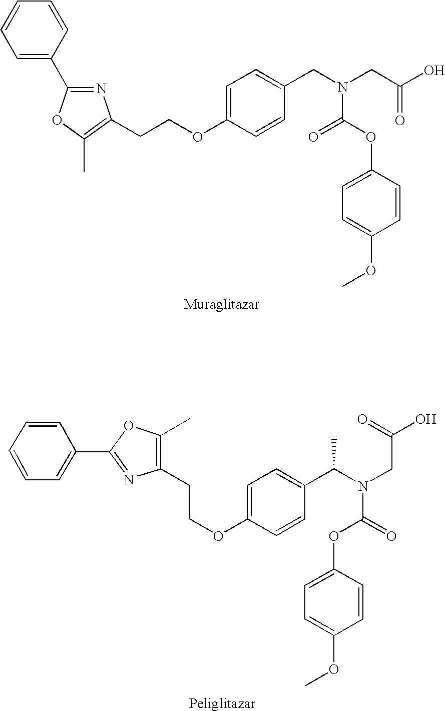

Murglitazar and Peliglitazar are discussed in WO-0121602 and U.S. patent application publication 2007/0015797. These references also disclose a genus of PPARγ agonists of formula:

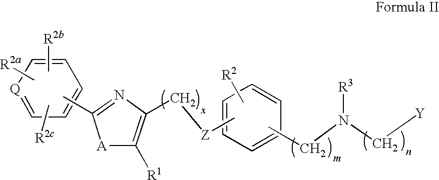

wherein:

x is 1, 2, 3, or 4; m is 1 or 2; n is 1 or 2;

Q is C or N;

A is O or S;

Z is O or a bond;

R¹ is H or alkyl;

X is CH or N;

R² is H, alkyl, alkoxy, halogen amino, or substituted amino;

R^{2a}, R^{2b} and R^{2c} are independently H, alkyl, alkoxy, halogen, amino, or substituted amino;

R³ is H, alkyl, arylalkyl, aryloxycarbonyl, alkyloxycarbonyl, alkynyloxycarbonyl, alkenyloxycarbonyl, arylcarbonyl, alkylcarbonyl, aryl, heteroaryl, alkyl(halo)aryloxycarbonyl, alkyloxy(halo)aryloxy-carbonyl, cycloalkylaryloxycarbonyl, cycloalkyloxyaryloxycarbonyl, cycloheteroalkyl, heteroarylcarbonyl, heteroaryl-heteroarylalkyl, alkylcarbonylamino, arylcarbonylamino, heteroarylcarbonylamino, alkoxycarbonylamino, aryloxycarbonylamino, heteroaryloxycarbonylamino, heteroaryl-heteroarylcarbonyl, alkylsulfonyl, alkenylsulfonyl, heteroaryloxycarbonyl, cycloheteroalkyloxycarbonyl, heteroarylalkyl, aminocarbonyl, substituted aminocarbonyl, alkylaminocarbonyl, arylaminocarbonyl, heteroarylalkenyl, cycloheteroalkyl-heteroarylalkyl; hydroxyalkyl, alkoxy, alkoxyaryloxycarbonyl, arylalkyloxycarbonyl, alkylaryloxycarbonyl, arylheteroarylalkyl, arylalkylarylalkyl, aryloxyarylalkyl, haloalkoxyaryloxycarbonyl, alkoxycarbonylaryloxycarbonyl, aryloxyaryloxycarbonyl, arylsulfinylarylcarbonyl, arylthioarylcarbonyl, alkoxycarbonylaryloxycarbonyl, arylalkenyloxycarbonyl, heteroaryloxyarylalkyl, aryloxyarylcarbonyl, aryloxyarylalkyloxycarbonyl, arylalkenyloxycarbonyl, arylalkylcarbonyl, aryloxyalkyloxycarbonyl, arylalkylsulfonyl, arylthiocarbonyl, arylalkenylsulfonyl, heteroarylsulfonyl, arylsulfonyl, alkoxyarylalkyl, heteroarylalkoxycarbonyl, arylheteroarylalkyl, alkoxyarylcarbonyl, aryloxyheteroarylalkyl, heteroarylalkyloxyarylalkyl, arylarylalkyl, arylalkenylarylalkyl, arylalkoxyarylalkyl, arylcarbonylarylalkyl, alkylaryloxyarylalkyl, arylalkoxycarbonylheteroarylalkyl, heteroarylarylalkyl, arylcarbonylheteroarylalkyl, heteroaryloxyarylalkyl, arylalkenylheteroarylalkyl, arylaminoarylalkyl, aminocarbonylarylarylalkyl;

Y is CO₂R⁴ (where R⁴ is H or alkyl, or a prodrug ester) or Y is a C-linked 1-tetrazole, a phosphinic acid of the structure P(O)(OR^{4a})R⁵, (where R^{4a} ia H or a prodrug ester, R⁵ is alkyl or aryl) or phosphonic acid of the structure P(O)(OR^{4a})₂, (where R^{4a} is H or a prodrug ester);

(CH₂)ₓ, (CH₂)ₙ, and (CH₂)ₘ may be optionally substituted with 1,2, or 3 substituents; including stereoisomers thereof, prodrug esters thereof, and pharmaceutically acceptable salts, hydrates, solvates, prodrugs, and polymorphs thereof, with the proviso that

where X is CH, A ia 0, Q is C, Z is O, and Y is CO₂R⁴, then R³ is other than H or alkyl containing 1 to 5 carbons in the normal chain;

or pharmaceutically acceptable salts, hydrates, solvates, polymorphs, or prodrugs thereof.

Reglitazar, PPARγ Agonist

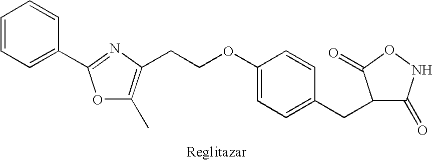

Reglitazar is discussed in WO-9518125 and U.S. Pat. No. 6,057,343. These references also disclose a genus of PPARγ agonists of formula:

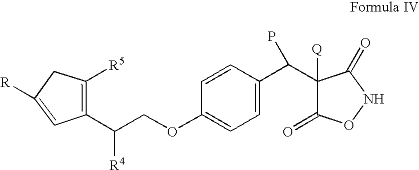

wherein:

R is an optionally substituted aromatic hydrocarbon, an optionally substituted alicyclic hydrocarbon, an optionally substituted heterocyclic group, an optionally substituted condensed heterocyclic group or a group of the formula:

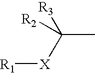

wherein R₁ is an optionally substituted aromatic hydrocarbon, an optionally substituted alicyclic hydrocarbon, an optionally substituted heterocyclic group or an optionally substituted condensed heterocyclic group, R₂ and R₃ are the same or different and each is a hydrogen atom or a lower alkyl, and X is an oxygen atom, a sulfur atom or a secondary amino;

R⁴ is a hydrogen atom, a lower alkyl or a hydroxy;

R⁵ is a lower alkyl optionally substituted by hydroxy; and

P and Q are each a hydrogen atom or P and Q together form a bond, or pharmaceutically acceptable salts, hydrates, solvates, or prodrugs thereof.

Naveglitazar, PPARγ Agonist

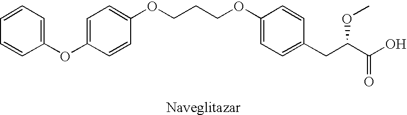

Naveglitazar is discussed in WO-02100403 and U.S. patent application publication 2005/0075378. These references also disclose a genus of PPARγ agonists of formula:

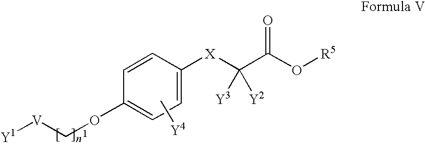

wherein:

n¹ is 2, 3, 4 or 5;

V is a bond or 0;

X is CH₂ or 0;

p is 0 or 1;

m is 1-4;

Y¹ is:

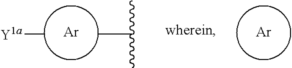

is aryl or heteroaryl, wherein aryl and heteroaryl are optionally substituted with one or more groups independently selected from the group consisting of hydrogen, C₁₋₆ alkyl, C₁₋₆ alkoxy, halo, haloalkyl and haloalkyloxy;

Y^{1a} is: hydrogen, (C₀₋₃) alkyl-aryl, C(O)-aryl, heteroaryl, cycloalkyl, heterocycloalkyl, aryloxy, NR⁵(CH₂)ₘOR⁵, aryl-Z-aryl, aryl-Z-heteroaryl, aryl-Z-cycloalkyl, aryl-Z-heterocycloalkyl, heteroaryl-Z-aryl, heteroaryl-Z-heterocycloalkyl or heterocycloalkyl-Z-aryl, wherein aryl, cycloalkyl, aryloxy, heteroaryl, and heterocycloalkyl are optionally substituted with one or more substituents independently selected from the group consisting of:

halo, hydroxyl, nitro, cyano, C₁₋₆ alkyl, C₁₋₆ alkoxy optionally substituted with N(R⁵)₂, haloalkyl, N(R⁵)₂, N[C(O)R⁵]₂, N[S(O)₂R⁵]₂, NR⁵S(O)₂R⁵, NR⁵C(O)R⁵, NR⁵C(O)O R⁵, C(O)N(R⁵)₂, C(O)O R⁵ and C(O)R⁵;

Z is a bond, -oxygen-, —C(O)NR⁵—, —NR⁵C(O)—, —NR⁵C(O)O—, —C(O)—, —NR⁵, —[O]p(CH₂)m—, —(CH₂)m[O]p—, —NR⁵(CH₂)m- or —(CH₂)mNR⁵—;

Y² and Y³ are each independently: hydrogen, C₁₋₆alkyl or C₁₋₆ alkoxy;

Y⁴ is: (C₁₋₃)alkyl-NR⁵C(O)—(C₀₋₅)alkyl-Y⁷—, (C₁₋₃)alkyl-NR⁵C(O)—(C₂₋₅)alkenyl-Y⁷, (C₁₋₃)alkyl-NR⁵C(O)—(C₂₋₅)alkynyl-Y⁷; (C₁₋₃)alkyl-NR⁵C(O)O—(C₀₋₅)alkyl-Y⁷, (C₁₋₃)alkyl-NR⁵C(O)NR⁵—(C₀₋₅)alkyl-Y⁷, (C₁₋₃)alkyl-NR⁵C(S)NR⁵—(C₀₋₅)alkyl-Y⁷, (C₀₋₃)alkyl-C(O)NR⁵—(C₀₋₅)alkyl-Y⁷, (C₀₋₃)alkyl-OC(O)NY¹⁰Y¹¹, (C₁₋₃)alkyl-NY¹⁰Y¹¹, (C₁₋₃)alky-O—(C₀₋₅)alkyl-Y⁷, (C₁₋₃)alkyl-S—(C₀₋₅)alkyl-Y⁷ or CN;

Y⁷ is: hydrogen, aryl, heteroaryl, C₁₋₁₂ alkyl, C₁₋₆ alkoxy, cycloalkyl, heterocycloalkyl, aryloxy, C(O)-heteroaryl or SR⁶,

wherein alkyl, aryl, aryloxy, alkoxy, heteroaryl, cycloalkyl, and heterocycloalkyl are optionally substituted with one or more groups independently selected from R⁷:

Y¹⁰ and Y¹¹ are each independently: hydrogen, aryl, heteroaryl, C₁₋₁₀alkyl, cycloalkyl, SO₂ (R⁶); or

Y¹⁰ and Y¹¹ together are a 5- to 10-membered heterocycloalkyl ring or heterocycloalkyl ring fused with aryl, and the heterocycloalkyl ring optionally containing one or more heteroatoms selected from N, O or S; and wherein, aryl, heteroaryl, heterocycloalkyl and alkyl are optionally substituted with one or more substituents independently selected from R⁷;

R⁵ is: hydrogen or C₁₋₆ alkyl;

R⁶ is: hydrogen, C₁₋₁₀ alkyl, cycloalkyl, aryl, or heteroaryl, wherein alkyl, cycloalkyl, aryl and heteroaryl are optionally substituted with one or more substituents independently selected from R⁷;

R⁷ is: halo, nitro, oxo, cyano, hydroxyl, benzyl, phenyl, phenoxy, heteroaryl, C(O)R⁶, C₁₋₁₀ alkyl, C₁₋₆ alkoxy, C₁₋₆ haloalkyl, C₁₋₆ haloalkyloxy, O(CH₂)m-phenyl, (CH₂)mOC(O)-aryl, C(O)OR⁵, S(O)₂R⁵, S(O)₂N(R⁵)₂, SR⁵ or N(R⁵)₂,

wherein phenyl and phenoxy are optionally substituted with one or more groups independently selected from halo or trifluoromethyl;

or pharmaceutically acceptable salts, hydrates, solvates, esters, or prodrugs thereof.

Oxeglitazar or EML-4156, PPARγ Agonist

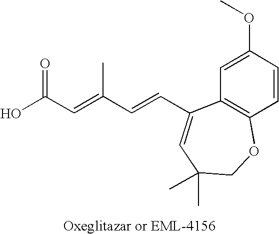

Oxeglitazar is discussed in WO-00039113 and WO-2004031166. These references also disclose a genus of PPARγ agonists of formula:

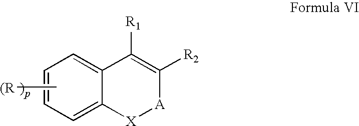

wherein:

X represents O or S;

A represents either the divalent radical —(CH₂)ₛ—CO—(CH₂)ₜ— or the divalent radical —(CH₂)ₛ-CR₃R₄—(CH₂)ₜ— in which radicals s=t=0 or else one of s and t has the value 0 and the other has the value 1;

R₄ represents a hydrogen atom or a (C₁-C₁₅)alkyl group;

R₁ and R₂ independently represent the Z chain defined below; a hydrogen atom; a (C₁-C₁₈)alkyl group; a (C₂-C₁₈)alkenyl group; a (C₂-C₁₈)alkynyl group; a (C₆-C₁₀)aryl group optionally substituted by a halogen atom, by an optionally halogenated (C₁-C₅)alkyl group or by an optionally halogenated (C₁-C₅)alkoxy group; or a mono- or bicyclic (C₄-C₁₂)heteroaryl group comprising one or more heteroatoms chosen from O, N and S which is optionally substituted by a halogen atom, by an optionally, halogenated (C₁-C₅)alkyl group or by an optionally halogenated (C₁-C₅)alkoxy group;

R₃ takes any one of meanings given above for R₁ and R₂, with the exception of the Z chain; or else

R₃ and R₄ together form a (C₂-C₆)alkylene chain optionally substituted by a halogen atom or by optionally halogenated (C₁-C₅)alkoxy;

R is chosen from a halogen atom; a cyano group; a nitro group; a carboxy group; an optionally halogenated (C₁-C₁₈)alkoxycarbonyl group; an Rₐ—CO—NH— or RₐR_{b}N—CO— group [in which Rₐ and R_{b} independently represent optionally halogenated (C₁-C₁₈)alkyl; a hydrogen atom; (C₆-C₁₀)aryl or (C₆-C₁₀)aryl(C₁-C₅)alkyl (where the aryl parts are optionally substituted by a halogen atom, by an optionally halogenated (C₁-C₅)alkyl group or by an optionally halogenated (C₁-C₅)alkoxy group); (C₃-C₁₂)cycloalkyl optionally substituted by a halogen atom, by an optionally halogenated (C₁-C₅)alkyl group or by an optionally halogenated (C₁-C₅)alkoxy group]; an optionally halogenated (C₁-C₁₈)alkyl group; optionally halogenated (C₁-C₁₈)alkoxy; and (C₆-C₁₀)aryl, (C₆-C₁₀)aryl(C₁-C₅)alkyl, (C₆-C₁₀)aryloxy, (C₃-C₁₂)cycloalkyl, (C₃-C₁₂)cycloalkenyl, (C₃-C₁₂)cycloalkyloxy, (C₃-C₁₂)cycloalkenyloxy or (C₆-C₁₀)aryloxycarbonyl in which the aryl, cycloalkyl and cycloalkenyl parts are optionally substituted by a halogen atom, by optionally halogenated (C₁-C₅)alkyl or by optionally halogenated (C₁-C₅)alkoxy;

p represents 0, 1, 2, 3 or 4;

Z represents the radical:

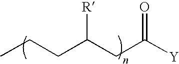

where n is 1 or 2;

the R′ groups independently represent a hydrogen atom; a (C₁-C₅)alkyl group; a (C₆-C₁₀)aryl group optionally substituted by a halogen atom, by an optionally halogenated (C₁-C₅)alkyl group or by optionally halogenated (C₁-C₅)alkoxy; or a mono- or bicyclic (C₄-C₁₂)heteroaryl group comprising one or more heteroatoms chosen from O, N and S which is optionally substituted by a halogen atom, by an optionally halogenated (C₁-C₅)alkyl group or by an optionally halogenated (C₁-C₅)alkoxy group;

Y represents —OH; (C₁-C₅)alkoxy; or the —NR_{c}R_{d} group (in which R_{c} and R_{d} independently represent a hydrogen atom; (C₁-C₅)alkyl; (C₃-C₈)cycloalkyl optionally substituted by a halogen atom, by optionally halogenated (C₁-C₅)alkyl or by optionally halogenated (C₁-C₅)alkoxy; (C₆-C₁₀)aryl optionally substituted by a halogen atom, by optionally halogenated (C₁-C₅)alkyl or by optionally halogenated (C₁-C₅)alkoxy; it being understood that one and one alone from R₁ and R₂ represents the Z chain;

or pharmaceutically acceptable salts, hydrates, solvates, polymorphs, or prodrugs thereof.

Imiglitazar, PPARγ Agonist

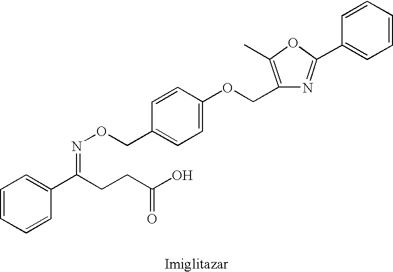

Imiglitazar is discussed in U.S. patent application publication 2003/0186985 These references also disclose a genus of PPARγ agonists of formula:

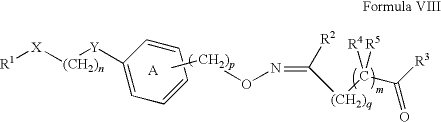

wherein:

R¹ is an optionally substituted hydrocarbon group, optionally substituted cyclic hydrocarbon group, or an optionally substituted heterocyclic group;

X is a bond, —CO—, —CH(OH)— or a group represented by —NR⁶— wherein R⁶ is a hydrogen atom or an optionally substituted alkyl group;

n is an integer of 1 to 3;

Y is an oxygen atom, a sulfur atom, —SO—, —SO₂— or a group represented by —NR⁷— wherein R⁷ is a hydrogen atom or an optionally substituted alkyl group;

a ring A is a benzene ring optionally having additional one to three substituents;

p is an integer of 1 to 8;

R² is a hydrogen atom, an optionally substituted hydrocarbon group or an optionally substituted heterocyclic group;

q is an integer of 0 to 6;

m is 0 or 1;

R³ is a hydroxy group, OR⁸ (R⁸ is an optionally substituted hydrocarbon group.) or NR⁹R¹⁰ (R⁹ and R¹⁰ are the same or different groups which are selected from a hydrogen atom, an optionally substituted hydrocarbon group, an optionally substituted heterocyclic group or an optionally substituted acyl group or R⁹ and R¹⁰ combine together to form a ring); R⁴ and R⁵ are the same or different groups which are selected from a hydrogen atom or an optionally substituted hydrocarbon group wherein R⁴ may form a ring with R²;

provided that when R¹ is a ethoxymethyl, a C₁₋₃ alkyl, phenyl or p-methoxyphenyl and q=m=O, R³ is NR⁹R¹⁰;

and provided that O-[2-chloro-4-(2-quinolylmethoxy)phenylmethyl]oxime of methyl pyruvate and [2-chloro-4-(2-quinolylmethoxy)phenylmethyl]-2-iminoxy-propionic acid are excluded;

or pharmaceutically acceptable salts, hydrates, solvates, polymorphs, or prodrugs thereof.

### Sipoglitazar or TAK-654, PPARγ Agonist

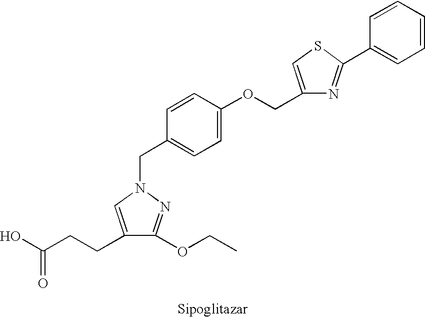

Sipoglitazar is discussed in WO-0134579 and EP 1,229,026 A1. These references also disclose a genus of PPARγ agonists of formula:

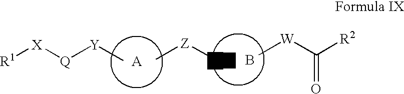

wherein:

R¹ is an optionally substituted 5-membered heterocyclic group;

X is a bond, an oxygen atom, a sulfur atom, —CO—, —CS—, —CR³(OR⁴)— or —NR⁵— (R³ is a hydrogen atom or an optionally substituted hydrocarbon group, R⁴ is a hydrogen atom or a hydroxy-protecting group and R⁵ is a hydrogen atom, an optionally substituted hydrocarbon group or an amino-protecting group);

Q is a divalent hydrocarbon group having 1 to 20 carbon atoms;

Y is a bond, an oxygen atom, a sulfur atom, —SO—, —SO₂—, —NR⁶—, —CONR⁶— or —NR⁶CO—(R⁶ is a hydrogen atom or an optionally substituted hydrocarbon group);

ring A is an aromatic ring optionally further having 1 to 3 substituents;

Z is —(CH₂)n-Z¹— or —Z¹—(CH₂)n- (n is an integer of 0 to 8, Z¹ is a bond, an oxygen atom, a sulfur atom, —SO—, —SO₂—, —NR⁷—, —CONR⁷— or —NR⁷CO— (R⁷ is a hydrogen atom or an optionally substituted hydrocarbon group));

ring B is a 5-membered heterocycle optionally further having 1 to 3 substituents;

W is a divalent saturated hydrocarbon group having 1 to 20 carbon atoms; and

R² is —OR⁸ (R⁸ is a hydrogen atom or an optionally substituted hydrocarbon group) or —N R⁹R¹⁰ (R⁹ and R¹⁰ are the same or different and each is a hydrogen atom, an optionally substituted hydrocarbon group, an optionally substituted heterocyclic group, or an acyl group, or R⁹ and R¹⁰ may be linked to form an optionally substituted ring together with the adjacent nitrogen atom),

provided that, when ring B is a nitrogen-containing 5-membered heterocycle, then the nitrogen-containing 5-membered heterocycle does not have, on the ring-constituting N atom, a substituent represented by the formula:

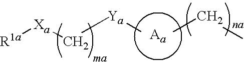

wherein

R^{1a} is an optionally substituted hydrocarbon group or an optionally substituted heterocyclic group;

Xa is a bond, an oxygen atom, a sulfur atom, —CO—, —CS—, —CR^{2a} (OR^{3a})— or —NR^{4a}— (R^{2a} and R^{4a} are each a hydrogen atom or an optionally substituted hydrocarbon group and R^{3a} is a hydrogen atom or a hydroxy-protecting group);

ma is an integer of 0 to 3;

Ya is an oxygen atom, a sulfur atom, —SO—, —SO₂—, —NR^{5a}—, CONR^{5a}— or —NR^{5a}CO—(R^{5a} is a hydrogen atom or an optionally substituted hydrocarbon group);

ring Aa is an aromatic ring optionally further having 1 to 3 substituents; and

na is an integer of 1 to 8,

or pharmaceutically acceptable salts, hydrates, solvates, polymorphs, or prodrugs thereof.

References cited in the preceding discussion and all other references cited in this application are hereby incorporated herein by reference in their entirety.

### SUMMARY

Neuropathic pain in mammals is treated by the administration of a therapeutically effective amount of an agonist of Peroxisome Proliferator-Activated Receptor gamma (PPARγ), wherein the agonist is a compound of one of Formulas I-IX.

An embodiment of the invention is a composition for the treatment of neuropathic pain comprising at least one agonist of the PPARγ or a salt, ester, hydrate, solvate, prodrug or polymorph thereof, incorporated in a pharmaceutically acceptable adjuvant, excipient, diluent or carrier composition, wherein the agonist is a compound of one of Formulas I-IX.

An embodiment of the invention is a method of treating neuropathic pain in a mammal in need of such treatment, comprising administering a therapeutically effective amount of an agonist of PPARγ or a salt, ester, hydrate, solvate, prodrug or polymorph thereof, wherein the agonist is a compound of one of Formulas I-IX.

An embodiment of the invention is a method of treating neuropathic pain in a mammal in need of such treatment comprising administering a therapeutically effective amount of a compound selected from the group consisting of Tesaglitazar, Muraglitazar, Peliglitazar, Farglitazar, Reglitazar, Naveglitazar, Oxeglitazar, Edaglitazone, Imiglitazar, Sipoglitazar and salts, hydrates, solvates, esters, prodrugs, and polymorphs thereof.

Another embodiment of the invention comprises compositions used for treating neuropathic pain comprising at least one compound selected from the group consisting of Tesaglitazar, Muraglitazar, Peliglitazar, Farglitazar, Reglitazar, Naveglitazar, Oxeglitazar, Edaglitazone, Imiglitazar, Sipoglitazar and salts, hydrates, solvates, esters, prodrugs, and polymorphs thereof, incorporated in a pharmaceutically acceptable adjuvant, excipient, diluent, or carrier composition.

Compounds of the invention may be administered in a variety of forms. These include, for example, solid, semi-solid and liquid dosage forms, such as tablets, pills, powders, liquid solutions or suspensions, liposomes, nasal/aerosolized dosage forms, implants, injectable and infusible solutions. Compounds may be used as their salts. Typical salts include lithium, sodium, potassium, aluminum, magnesium, calcium, zinc, manganese, ammonium salts and the like and mixtures thereof. In addition, salts may include salts formed with acids such as organic acids or inorganic acids. Typical acids used to form salts may include HF, HCl, HBr, HI, sulfuric, perchloric, phosphoric, acetic, formic, propionic, butyric, pentanoic, benzoic, and the like.

The active compounds can be administered alone or in combination with pharmaceutically acceptable carriers or diluents by any of several routes. More particularly, the active compounds can be administered in a wide variety of different dosage forms, e.g., they may be combined with various pharmaceutically accentable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in either sesame or peanut oil or in aqueous propylene glycol can be employed. The aqueous solutions should be suitably buffered, if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active compounds topically and this can be done by way of creams, a patch, jells, gels, pastes, ointments and the like, in accordance with standard pharmaceutical practice.

The dosage of a specific active compound of the invention depends upon many factors that are well known to those skilled in the art, for example: the particular compound; the condition being treated; the age, weight, and clinical condition of the recipient patient; and the experience and judgment of the clinician or practitioner administering the therapy. An effective amount of the compound is that which provides either subjective relief of symptoms or an objectively identifiable improvement as noted by the clinician or other qualified observer. The dosing range varies with the compound used, the route of administration and the potency of the particular compound.

### DESCRIPTION OF THE FIGURES

FIG. 1—Figure from patent publication WO2006085686 titled, “Remedy for neurogenic pain”, wherein the invention intends “ . . . to provide a remedy for neurogenic pain which contains, as the active ingredient, a PPAR antagonist . . . .” The inventors are Tanabe & Tsutomu from the Tokyo Medical & Dental University. After administration of GW9662, a PPAR antagonist, at 3 or 30 mg/kg, the threshold (in g) was increased, indicating a decrease in pain.

FIG. 2—Figure from journal publication titled, “Thiazolidinedione Class of PPARγ Agonists Prevent Neuronal Damage, Motor Dysfunction, Myelin loss, Neuropathic Pain and Inflammation Following Spinal Cord Injury in Adult Rats” (J. Pharmacol. Exp. Ther. 2007. 320:1002-12). Effect of pioglitazone on long-term motor recovery and neuropathic pain: In a cohort of rats subjected to spinal cord injury (SCI), pioglitazone treatment induced a sustained improved recovery of motor function compared to vehicle treatment. The BBB scores were significantly higher at all the time points after SCI (3 to 42 days) in the pioglitazone group over vehicle group (A). Pretreating rats with the PPARγ antagonist GW9662 completely abolished the improved motor function recovery induced by pioglitazone after SCI (B). At 28 days after SCI, the vehicle treated rats showed a significant decrease over baseline in the latency in withdrawing the paw from the heated source indicating neuropathic pain (C). Pioglitazone treated rats subjected to SCI showed no change in the thermal delay over baseline indicating the ease of neuropathic pain (C). In rats treated with GW9662 before treating with pioglitazone, the thermal latency was similar to that observed in the vehicle group (C). The values in panels A to C are mean±SD (n=6 rats/group). Statistics: *p<0.05 compared with the vehicle control (panel A), ap<0.05 compared with the baseline and bp<0.05 compared with the vehicle group (panel C) by ANOVA followed by Tukey-Kramer multiple comparisons post-test.

### DETAILED DESCRIPTION

Embodiments of the invention provide methods for treating neuropathic pain by the administration of a therapeutically effective amount of an agonist of PPARγ.

According to embodiments of the invention, a therapeutically effective amount of a compound that agonizes PPARγ is administered to a subject to treat neuropathic pain. A compound useful in carrying out a therapeutic method embodiment of the invention is advantageously formulated in a pharmaceutical composition in combination with a pharmaceutically acceptable carrier. The amount of compound in the pharmaceutical composition depends on the desired dosage and route of administration. In one embodiment, suitable dose ranges of the active ingredient are from about 0.01 mg/kg to about 1500 mg/kg of body weight taken at necessary intervals (e.g., daily, every 12 hours, etc.). In another embodiment, a suitable dosage range of the active ingredient is from about 0.2 mg/kg to about 150 mg/kg of body weight taken at necessary intervals. In another embodiment, a suitable dosage range of the active ingredient is from about 1 mg/kg to about 15 mg/kg of body weight taken at necessary intervals.

In one embodiment of the method of treating neuropathic pain, the dosage and administration are such that PPARγ is only partially inhibited so as to avoid any unacceptably deleterious effects.

A therapeutically effective compound can be provided to the subject in a standard formulation that includes one or more pharmaceutically acceptable additives, such as excipients, lubricants, diluents, flavorants, colorants, buffers, and disintegrants. The formulation may be produced in unit dosage from for administration by oral, parenteral, transmucosal, intranasal, rectal, vaginal, or transdermal routes. Parenteral routes include intravenous, intra-arterial, intramuscular, intradermal subcutaneous, intraperitoneal, intraventricular, intrathecal, and intracranial administration.

The pharmaceutical composition can be added to a retained physiological fluid such as blood or synovial fluid. In one embodiment for CNS administration, a variety of techniques are available for promoting transfer of the therapeutic agent across the blood brain barrier, or to gain entry into an appropriate cell, including disruption by surgery or injection, co-administration of a drug that transiently opens adhesion contacts between CNS vasculature endothelial cells, and co-administration of a substance that facilitates translocation through such cells. In another embodiment, for example, to target the peripheral nervous system (PNS), the pharmaceutical composition has a restricted ability to cross the blood brain barrier and can be administered using techniques known in the art.

In another embodiment of the method of treating neuropathic pain, the agonist of PPARγ is delivered in a vesicle, particularly a liposome. In one embodiment, the agonist of PPARγ is delivered topically (e.g., in a cream) to the site of pain (or related disorder) to avoid the systemic effects of agonizing PPARγ in non-target cells or tissues.

In another embodiment of the method of treating neuropathic pain, the therapeutic agent is delivered in a controlled release manner. For example, a therapeutic agent can be administered using intravenous infusion with a continuous pump, or in a polymer matrix such as poly-lactic/glutamic acid (PLGA), or in a pellet containing a mixture of cholesterol and the active ingredient, or by subcutaneous implantation, or by transdermal patch.

Three independent microarray studies, Chiang et al (patent publication WO 2005/014849 A2), Valder et al (Neurochem, 2003. 87:560), and Wang et al (Neuroscience, 2002. 114:529), were reported for the rat spinal nerve ligation (SNL) model of neuropathic pain. Each of the three groups performed gene expression analysis using the Affymetrix platform on RNA extracted from dorsal root ganglia tissue isolated from rats subjected to SNL. The information on genes reported as regulated by significance criteria specific to each study were combined using Aestus Therapeutics Inc (ATx) proprietary methods. By combining the three datasets for analysis and applying ATx multidimensional analysis, a physiological function previously unreported as important for neuropathic pain was identified—agonism of PPARγ.

Tesaglitazar. PPARγ Agonist/PPARα Agonist

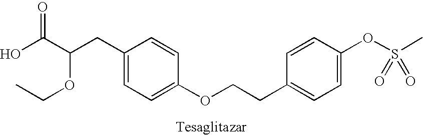

An embodiment of the invention comprises a method of treating neuropathic pain comprising treating a mammal in need such treatment with a therapeutically effective amount of Tesaglitazar, and pharmaceutically acceptable salts, hydrates, solvates, prodrugs, and polymorphs thereof.

Edaglitazone. PPARγ Agonist.

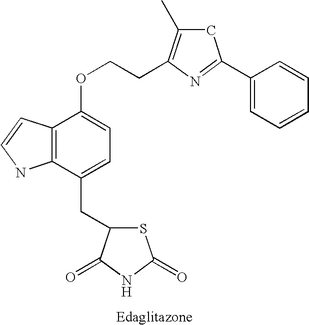

An embodiment of the invention is a method of treating neuropathic pain comprising treating a mammal in need of such treatment with a therapeutically effective amount of Edaglitazone, or pharmaceutically acceptable salts, hydrates, solvates, polymorphs, or prodrugs thereof.

An embodiment of the invention is a method of treating neuropathic pain comprising treating a mammal in need of such treatment with a therapeutically effective amount of a compound of the formula:

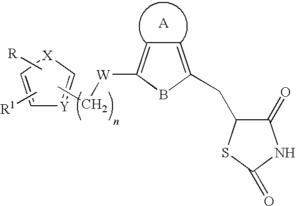

wherein:
A is a carbocyclic ring with 5 or 6 carbon atoms or a heterocyclic ring with a maximum of 4 heteroatoms in which the heteroatoms can be the same or different and denote oxygen, nitrogen, or sulfur and the heterocycles can if desired, carry an oxygen atom on one or several nitrogen atoms;
B is —CH═CH—, —N═CH—, —CH═N—, O, or S;
W is CH2, OCH(OH), CO or —CH═CH—;
X is S, O, or NR2 in which the residue R2 is hydrogen or C1-6 alkyl;
Y is CH or N;
R is naphthyl, pyridyl, furyl, thienyl, or phenyl which if desired is mono- or disubstituted with C1-3 alkyl, CF3, C1-3 alkoxy, F, Cl, or Br;
R1 is hydrogen or C1-6 alkyl;
n is 1 to 3; and

tautomers, enantiomers, diasteromers, and pharmaceutically acceptable salts, hydrates, solvates, prodrugs, and polymorphs thereof.

Farglitazar. PPARγ Agonist; Retinoid X Receptor Modulator.

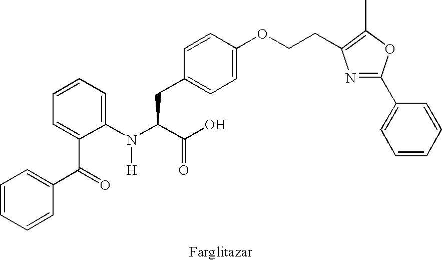

An embodiment of the invention is a method of treating neuropathic pain comprising treating a mammal in need of such treatment with a therapeutically effective amount of Farglitazar, or pharmaceutically acceptable salts, hydrates, solvates, polymorphs, or prodrugs thereof.

An embodiment of the invention is a method of treating neuropathic pain comprising treating a mammal in need of such treatment with a therapeutically effective amount of a compound of the formula:

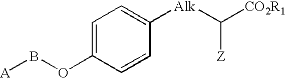

wherein:

A is selected from the group consisting of:
(i) phenyl, wherein the phenyl is optionally substituted by one or more of the following groups: halogen atoms, C₁₋₆ alkyl, C₁₋₃ alkoxy, C1-3 fluoroalkoxy, nitrile, or —NR⁷R⁸ where R⁷ and R⁸ are independently hydrogen or C₁₋₃ alkyl;
(ii) a 5- or 6-membered heterocyclic group containing at least one heteroatom selected from oxygen, nitrogen and sulfur; and
(iii) a fused bicyclic ring

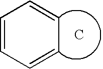

wherein ring C represents a heterocyclic group as defined in point (ii) above, which bicyclic ring is attached to group B via a ring atom of C;

B is selected from the group consisting of:
(iv) C₁₋₆ alkene;
(v) -MC₁₋₆ alkene or C₁₋₆ alkeneMC₁₋₆ alkene, wherein M is O, S, or —NR² wherein R² represents hydrogen or C₁₋₃ alkyl;
(vi) a 5- or 6-membered heterocyclic group containing at least one nitrogen heteroatom and optionally at least one further heteroatom selected from oxygen, nitrogen and sulfur and optionally substituted by C₁₋₃ alkyl; and
(vii) Het-C₁₋₆ alkylene, wherein Het represents a heterocyclic group as defined in point (vi) above;

Alk represents C₁₋₃ alkylene;

R1 represents hydrogen or C₁₋₃ alkyl;

Z is selected from the group consisting of:
(viii) —(C₁₋₃alkylene)phenyl, which phenyl is optionally substituted by one or more halogen atoms; and
(ix)-NR³R⁴, wherein R³ represents hydrogen or C₁₋₃alkyl, and R⁴ represents —Y—(C═O)-T-R⁵, or —Y—(CH(OH))-T-R⁵, wherein:
   (a) Y represents a bond, C₁₋₆ alkylene, C₂₋₆alkenylene, C₄₋₆ cycloalkene or cycloalkenylene, a heterocyclic group as defined in point (vi) above, or phenyl optionally substituted by one or more C₁₋₃ alkyl groups and/or one or more halogen atoms;
   (b) T represents a bond, C₁₋₃ alkyleneoxy, —O— or —N(R⁶)—, wherein R6 represents hydrogen or C₁₋₃ alkyl;
   (c) R⁵ represents C₁₋₆ alkyl, C₄₋₆ cycloalkyl or cycloalkenyl, phenyl (optionally substituted by one or more of the following groups; halogen atoms, C₁₋₃ alkyl, C₁₋₃ alkoxy groups, C₀₋₃ alkyleneNR⁹R¹⁰ (where each R⁹ and R¹⁰ is independently hydrogen, C₁₋₃ alkyl, —SO₂C₁₋₃alkyl, or —CO₂C₁₋₃ alkyl, —SO2NHC₁₋₃alkyl), C₀₋₃ alkyleneCO₂H, C₀₋₃alkyleneCO₂C₁₋₃alkyl, or —OCO₂C(O)NH₂), a 5- or 6-membered heterocyclic group as defined in point (ii) above, a bicyclic fused ring

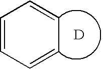

wherein ring D represents a 5- or 6-membered heterocyclic group containing at least one heteroatom selected from oxygen, nitrogen and sulfur and optionally substituted by (═O), which bicyclic ring is attach to T vi a ring atom of ring D: or —C₁₋₆ alkyleneMR¹¹; M is O, S, or NR¹² wherein R¹² and R¹¹ are independently hydrogen or C₁₋₃ alkyl; or a tautomeric form thereof, and/or a pharmaceutically acceptable salt, hydrate, solvate, or polymorph thereof.

The terms C₁₋₃ alkyl or alkylene and C₁₋₆ alkyl or alkylene as used herein respectively contain 1 to 3 or 1 to 6 carbon atoms and appropriately include straight chained and branched alkyl or alkylene groups, typically methyl, methylene, ethyl and ethylene groups, and straight chained and branched propyl, propylene, butyl and butylene groups. The term C₂₋₆ alkenyl or alkenylene as used herein contains 2 to 6 carbon atoms and appropriately includes straight chained and branched alkenyl and alkenylene groups, in particular propenylene or the like;

and pharmaceutically acceptable salts, hydrates, solvates, polymorphs, or prodrugs thereof.

Muraglitazar

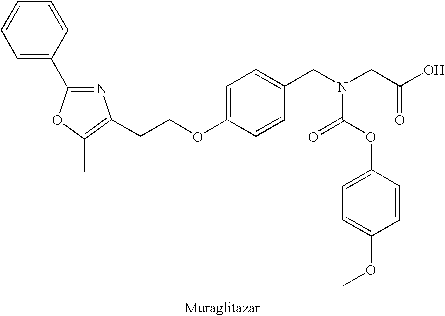

An embodiment of the invention is a method of treating neuropathic pain comprising treating a mammal in need of such treatment with a therapeutically effective amount of Murglitazar, or pharmaceutically acceptable salts, hydrates, solvates, polymorphs, or prodrugs thereof.

An embodiment of the invention is a method of treating neuropathic pain comprising treating a mammal in need of such treatment with a therapeutically effective amount of a compound of formula:

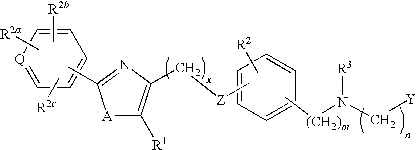

wherein x is 1, 2, 3, or 4; m is 1 or 2; n is 1 or 2;

Q is C or N;

A is O or S;

Z is O or a bond;

R¹ is H or alkyl;

X is CH or N;

R² is H, alkyl, alkoxy, halogen amino, or substituted amino;

R^{2a}, R^{2b}, and R^{2c} are independently H, alkyl, alkoxy, halogen, amino, or substituted amino;

R³ is H, alkyl, arylalkyl, aryloxycarbonyl, alkyloxycarbonyl, alkynyloxycarbonyl, alkenyloxycarbonyl, arylcarbonyl, alkylcarbonyl, aryl, heteroaryl, alkyl(halo)aryloxycarbonyl, alkyloxy(halo)aryloxy-carbonyl, cycloalkylaryloxycarbonyl, cycloalkyloxyaryloxycarbonyl, cycloheteroalkyl, heteroarylcarbonyl, heteroaryl-heteroarylalkyl, alkylcarbonylamino, arylcarbonylamino, heteroarylcarbonylamino, alkoxycarbonylamino, aryloxycarbonylamino, heteroaryloxycarbonylamino, heteroaryl-heteroarylcarbonyl, alkylsulfonyl, alkenylsulfonyl, heteroaryloxycarbonyl, cycloheteroalkyloxycarbonyl, heteroarylalkyl, aminocarbonyl, substituted aminocarbonyl, alkylaminocarbonyl, arylaminocarbonyl, heteroarylalkenyl, cycloheteroalkyl-heteroarylalkyl; hydroxyalkyl, alkoxy, alkoxyaryloxycarbonyl, arylalkyloxycarbonyl, alkylaryloxycarbonyl, arylheteroarylalkyl, arylalkylarylalkyl, aryloxyarylalkyl, haloalkoxyaryloxycarbonyl, alkoxycarbonylaryloxycarbonyl, aryloxyaryloxycarbonyl, arylsulfinylarylcarbonyl, arylthioarylcarbonyl, alkoxycarbonylaryloxycarbonyl, arylalkenyloxycarbonyl, heteroaryloxyarylalkyl, aryloxyarylcarbonyl, aryloxyarylalkyloxycarbonyl, arylalkenyloxycarbonyl, arylalkylcarbonyl, aryloxyalkyloxycarbonyl, arylalkylsulfonyl, arylthiocarbonyl, arylalkenylsulfonyl, heteroarylsulfonyl, arylsulfonyl, alkoxyarylalkyl, heteroarylalkoxycarbonyl, arylheteroarylalkyl, alkoxyarylcarbonyl, aryloxyheteroarylalkyl, heteroarylalkyloxyarylalkyl, arylarylalkyl, arylalkenylarylalkyl, arylalkoxyarylalkyl, arylcarbonylarylalkyl, alkylaryloxyarylalkyl, arylalkoxycarbonylheteroarylalkyl, heteroarylarylalkyl, arylcarbonylheteroarylalkyl, heteroaryloxyarylalkyl, arylalkenylheteroarylalkyl, arylaminoarylalkyl, aminocarbonylarylarylalkyl;

Y is CO₂R⁴ (where R⁴ is H or alkyl, or a prodrug ester) or Y is a C-linked 1-tetrazole, a phosphinic acid of the structure P(O)(OR^{4a})R⁵, (where R^{4a} ia H or a prodrug ester, R⁵ is alkyl or aryl) or phosphonic acid of the structure P(O)(OR^{4a})₂, (where R^{4a} is H or a prodrug ester);

(CH₂)ₓ, (CH₂)—, and (CH₂)ₘ may be optionally substituted with 1, 2, or 3 substituents;

including stereoisomers thereof, prodrug esters thereof, and pharmaceutically acceptable salts, hydrates, solvates, prodrugs, and polymorphs thereof, with the proviso that

where X is CH, A ia O, Q is C, Z is O, and Y is CO₂R⁴, then R³ is other than H or alkyl containing 1 to 5 carbons in the normal chain;

or pharmaceutically acceptable salts, hydrates, solvates, polymorphs, or prodrugs thereof.

Peliglitazar

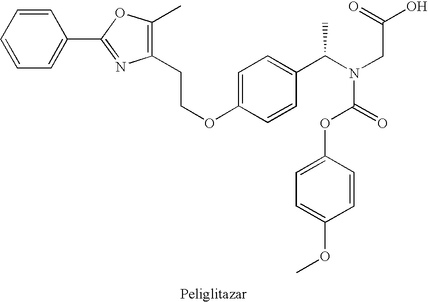

An embodiment of the invention is a method of treating neuropathic pain comprising treating a mammal in need of such treatment with a therapeutically effective amount of Peliglitazar, or pharmaceutically acceptable salts, hydrates, solvates, polymorphs, or prodrugs thereof.

Reglitazar

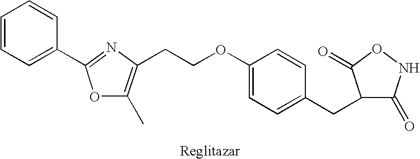

An embodiment of the invention is a method of treating neuropathic pain comprising treating a mammal in need of such treatment with a therapeutically effective amount of Reglitazar, or pharmaceutically acceptable salts, hydrates, solvates, polymorphs, or prodrugs thereof.

An embodiment of the invention is a method of treating neuropathic pain comprising treating a mammal in need of such treatment with a therapeutically effective amount of a compound of formula:

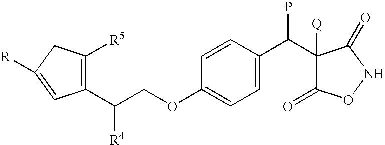

wherein

wherein R is an optionally substituted aromatic hydrocarbon, an optionally substituted alicyclic hydrocarbon, an optionally substituted heterocyclic group, an optionally substituted condensed heterocyclic group or a group of the formula:

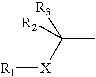

wherein R₁ is an optionally substituted aromatic hydrocarbon, an optionally substituted alicyclic hydrocarbon, an optionally substituted heterocyclic group or an optionally substituted condensed heterocyclic group, R₂ and R₃ are the same or different and each is a hydrogen atom or a lower alkyl, and X is an oxygen atom, a sulfur atom or a secondary amino;

R⁴ is a hydrogen atom, a lower alkyl or a hydroxy;

R⁵ is a lower alkyl optionally substituted by hydroxy; and

P and Q are each a hydrogen atom or P and Q together form a bond, or pharmaceutically acceptable salts, hydrates, solvates, polymorphs, or prodrugs thereof.

Naveglitazar

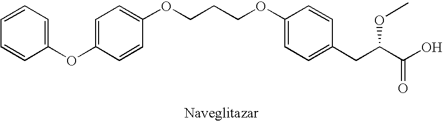

An embodiment of the invention is a method of treating neuropathic pain comprising treating a mammal in need of such treatment with a therapeutically effective amount of Naveglitazar, or a pharmaceutically acceptable salt, hydrate, solvate, or polymorph thereof.

An embodiment of the invention is a method of treating neuropathic pain comprising treating a mammal in need of such treatment with a therapeutically effective amount of a compound of formula:

wherein:

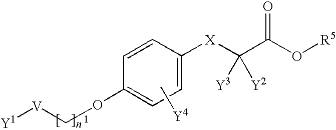

n¹ is 2, 3, 4 or 5;

V is a bond or O;

X is CH₂ or O;

p is 0 or 1;

m is 1-4;

Y^{1a} is:

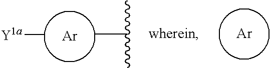

is: aryl or heteroaryl,

wherein aryl and heteroaryl are optionally substituted with one or more groups independently selected from the group consisting of:

hydrogen, C₁₋₆ alkyl, C₁₋₆ alkoxy, halo, haloalkyl and haloalkyloxy;

Y^{1a} is: hydrogen, (C₀₋₃)alkyl-aryl, C(O)-aryl, heteroaryl, cycloalkyl, heterocycloalkyl, aryloxy, NR⁵(CH₂)ₘOR⁵, aryl-Z-aryl, aryl-Z-heteroaryl, aryl-Z-cycloalkyl, aryl-Z-heterocycloalkyl, heteroaryl-Z-aryl, heteroaryl-Z-heterocycloalkyl or heterocycloalkyl-Z-aryl,

wherein aryl, cycloalkyl, aryloxy, heteroaryl, and heterocycloalkyl are optionally substituted with one or more substituents independently selected from the group consisting of:

halo, hydroxyl, nitro, cyano, C₁₋₆ alkyl, C₁₋₆ alkoxy optionally substituted with N(R⁵)₂, haloalkyl, N(R⁵)₂, N[C(O)R⁵]₂, N[S(O)₂R⁵]₂, NR⁵S(O)₂R⁵, NR⁵C(O)R⁵, NR⁵C(O)O R⁵, C(O)N(R⁵)₂, C(O)O R⁵ and C(O)R⁵;

Z is: a bond, -oxygen-, —C(O)NR⁵—, —NR⁵C(O)—, —NR⁵C(O)O—, —C(O)—, —NR⁵, —[O]p(CH₂)m-, —(CH₂)m[O]p—, —NR⁵(CH₂)m- or —(CH₂)mNR⁵—;

Y² and Y³ are each independently: hydrogen, C₁₋₆alkyl or C₁₋₆ alkoxy;

Y⁴ is: (C₁₋₃)alkyl-NR⁵C(O)—(C₀₋₅)alkyl-Y⁷—(C₁₋₃)alkyl-NR⁵C(O)—(C₂₋₅)alkenyl-Y⁷, (C₁₋₃)alkyl-NR⁵C(O)—(C₂₋₅)alkynyl-Y⁷; (C₁₋₃)alkyl-NR⁵C(O)O—(C₀₋₅)alkyl-Y⁷, (C₁₋₃)alkyl-NR⁵C(O)NR⁵—(C₀₋₅)alkyl-Y⁷, (C₁₋₃)alkyl-NR⁵C(S)NR⁵—(C₀₋₅)alkyl-Y⁷, (C₀₋₃)alkyl-C(O)NR⁵—(C₀₋₅)alkyl-Y⁷, (C₀₋₃)alkyl-OC(O)NY¹⁰Y¹¹, (C₁₋₃)alkyl-NY¹⁰Y¹¹, (C₁₋₃)alkyl-O—(C₀₋₅)alkyl-Y⁷, (C₁₋₃)alkyl-S—(C₀₋₅)alkyl-Y⁷ or CN;

Y⁷ is: hydrogen, aryl, heteroaryl, C₁₋₁₂ alkyl, C₁₋₆ alkoxy, cycloalkyl, heterocycloalkyl, aryloxy, C(O)-heteroaryl or SR⁶,

wherein alkyl, aryl, aryloxy, alkoxy, heteroaryl, cycloalkyl, and heterocycloalkyl are optionally substituted with one or more groups independently selected from R⁷:

Y¹⁰ and Y¹¹ are each independently: hydrogen, aryl, heteroaryl, C₁-C₁₀alkyl, cycloalkyl, SO₂(R⁶); or

Y¹⁰ and Y¹¹ together are a 5- to 10-membered heterocycloalkyl ring or heterocycloalkyl ring fused with aryl, and the heterocycloalkyl ring optionally containing one or more heteroatoms selected from N, O or S; and wherein, aryl, heteroaryl, heterocycloalkyl and alkyl are optionally substituted with one or more substituents independently selected from R⁷;

R⁵ is: hydrogen or C₁₋₆ alkyl;

R⁶ is: hydrogen, C₁₋₁₀ alkyl, cycloalkyl, aryl, or heteroaryl, wherein alkyl, cycloalkyl, aryl and heteroaryl are optionally substituted with one or more substituents independently selected from R⁷;

R⁷ is: halo, nitro, oxo, cyano, hydroxyl, benzyl, phenyl, phenoxy, heteroaryl, C(O)R⁶, C₁₋₁₀alkyl, C₁₋₆ alkoxy, C₁₋₆ haloalkyl, C₁₋₆ haloalkyloxy, O(CH₂)m -phenyl, (CH₂)mOC(O)-aryl, C(O)OR⁵, S(O)₂R⁵, S(O)₂N(R⁵)₂, SR⁵ or N(R⁵)₂,

wherein phenyl and phenoxy are optionally substituted with one or more groups independently selected from halo or trifluoromethyl;

or pharmaceutically acceptable salts, hydrates, solvates, polymorphs, or prodrugs thereof.

Oxeglitazar or EML-4156

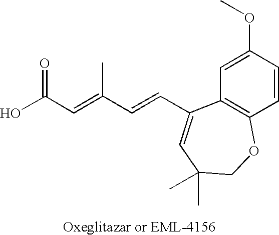

An embodiment of the invention is a method of treating neuropathic pain comprising treating a mammal in need of such treatment with a therapeutically effective amount of Oxeglitazar, or pharmaceutically acceptable salts, hydrates, solvates, polymorphs, or prodrugs thereof.

An embodiment of the invention is a method of treating neuropathic pain comprising treating a mammal in need of such treatment with a therapeutically effective amount of a compound of formula:

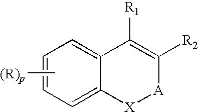

wherein: X represents O or S;

A represents either the divalent radical —(CH₂)ₛ—CO—(CH₂)ₜ— or the divalent radical —(CH₂)ₛ—CR₃R₄—(CH₂)ₜ— in which radicals s=t=0 or else one of s and t has the value 0 and the other has the value 1;

R₄ represents a hydrogen atom or a (C₁-C₁₅)alkyl group;

R₁ and R₂ independently represent the Z chain defined below; a hydrogen atom; a (C₁-C₁₈)alkyl group; a (C₂-C₁₈)alkenyl group; a (C₂-C₁₈)alkynyl group; a (C₆-C₁₀)aryl group optionally substituted by a halogen atom, by an optionally halogenated (C₁-C₅)alkyl group or by an optionally halogenated (C₁-C₅)alkoxy group; or a mono- or bicyclic (C₄-C₁₂)heteroaryl group comprising one or more heteroatoms chosen from O, N and S which is optionally substituted by a halogen atom, by an optionally, halogenated (C₁-C₅)alkyl group or by an optionally halogenated (C₁-C₅)alkoxy group;

R₃ takes any one of meanings given above for R₁ and R₂, with the exception of the Z chain; or else

R₃ and R₄ together form a (C₂-C₆)alkylene chain optionally substituted by a halogen atom or by optionally halogenated (C₁-C₅)alkoxy;

R is chosen from a halogen atom; a cyano group; a nitro group; a carboxy group; an optionally halogenated (C₁-C₁₈)alkoxycarbonyl group; an Rₐ—CO—NH— or RₐR_{b} N—CO— group [in which Rₐ and R_{b} independently represent optionally halogenated (C₁-C₁₈)alkyl; a hydrogen atom; (C₆-C₁₀)aryl or (C₆-C₁₀)aryl(C₁-C₅)alkyl (where the aryl parts are optionally substituted by a halogen atom, by an optionally halogenated (C₁-C₅)alkyl group or by an optionally halogenated (C₁-C₅)alkoxy group); (C₃-C₁₂)cycloalkyl optionally substituted by a halogen atom, by an optionally halogenated (C₁-C₅)alkyl group or by an optionally halogenated (C₁-C₅)alkoxy group]; an optionally halogenated (C₁-C₁₈)alkyl group; optionally halogenated (C₁-C₁₈)alkoxy; and (C₆-C₁₀)aryl, (C₆-C₁₀)aryl(C₁-C₅)alkyl, (C₆-C₁₀)aryloxy, (C₃-C₁₂)cycloalkyl, (C₃-C₁₂)cycloalkenyl, (C₃-C₁₂)cycloalkyloxy, (C₃-C₁₂)cycloalkenyloxy or (C₆-C₁₀)aryloxycarbonyl in which the aryl, cycloalkyl and cycloalkenyl parts are optionally substituted by a halogen atom, by optionally halogenated (C₁-C₅)alkyl or by optionally halogenated (C₁-C₅)alkoxy;

p represents 0, 1, 2, 3 or 4;

Z represents the radical:

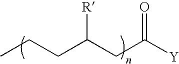

where n is 1 or 2;

the R′ groups independently represent a hydrogen atom; a (C₁-C₅)alkyl group; a (C₆-C₁₀)aryl group optionally substituted by a halogen atom, by an optionally halogenated (C₁-C₅)alkyl group or by optionally halogenated (C₁-C₅)alkoxy; or a mono- or bicyclic (C₄-C₁₂)heteroaryl group comprising one or more heteroatoms chosen from O, N and S which is optionally substituted by a halogen atom, by an optionally halogenated (C₁-C₅)alkyl group or by an optionally halogenated (C₁-C₅)alkoxy group;

Y represents —OH; (C₁-C₅)alkoxy; or the —NR_{c}R_{d} group (in which R_{c} and R_{d} independently represent a hydrogen atom; (C₁-C₅)alkyl; (C₃-C₈)cycloalkyl optionally substituted by a halogen atom, by optionally halogenated (C₁-C₅)alkyl or by optionally halogenated (C₁-C₅)alkoxy; (C₆-C₁₀)aryl optionally substituted by a halogen atom, by optionally halogenated (C₁-C₅)alkyl or by optionally halogenated (C₁-C₅)alkoxy; it being understood that one and one alone from R₁ and R₂ represents the Z chain;

or pharmaceutically acceptable salts, hydrates, solvates, polymorphs, or prodrugs thereof.

Imiglitazar

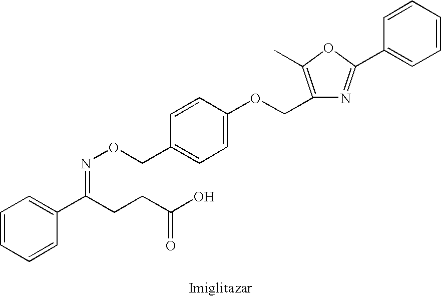

An embodiment of the invention is a method of treating neuropathic pain comprising treating a mammal in need of such treatment with a therapeutically effective amount of Imiglitazar, or pharmaceutically acceptable salts, hydrates, solvates, polymorphs, or prodrugs thereof.

An embodiment of the invention is a method of treating neuropathic pain comprising treating a mammal in need of such treatment with a therapeutically effective amount of a compound of formula:

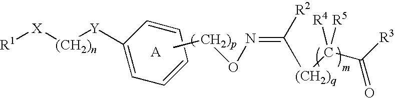

wherein:

wherein R¹ is an optionally substituted hydrocarbon group, optionally substituted cyclic hydrocarbon group, or an optionally substituted heterocyclic group;

X is a bond, —CO—, —CH(OH)— or a group represented by —NR⁶— wherein R⁶ is a hydrogen atom or an optionally substituted alkyl group;

n is an integer of 1 to 3;

Y is an oxygen atom, a sulfur atom, —SO—, —SO₂— or a group represented by —NR⁷— wherein R⁷ is a hydrogen atom or an optionally substituted alkyl group;

a ring A is a benzene ring optionally having additional one to three substituents;

p is an integer of 1 to 8;

R² is a hydrogen atom, an optionally substituted hydrocarbon group or an optionally substituted heterocyclic group;

q is an integer of 0 to 6;

m is 0 or 1;

R³ is a hydroxy group, OR⁸ (R⁸ is an optionally substituted hydrocarbon group.) or NR⁹R¹⁰(R⁹ and R¹⁰ are the same or different groups which are selected from a hydrogen atom, an optionally substituted hydrocarbon group, an optionally substituted heterocyclic group or an optionally substituted acyl group or R⁹ and R¹⁰ combine together to form a ring); R⁴ and R⁵ are the same or different groups which are selected from a hydrogen atom or an optionally substituted hydrocarbon group wherein R⁴ may form a ring with R²;

provided that when R¹ is a ethoxymethyl, a C₁₋₃ alkyl, phenyl or p-methoxyphenyl and q=m=O, R³ is NR⁹R¹⁰;

and provided that O-[2-chloro-4-(2-quinolylmethoxy)phenylmethyl]oxime of methyl pyruvate and [2-chloro-4-(2-quinolylmethoxy)phenylmethyl]-2-iminoxy-propionic acid are excluded;

or pharmaceutically acceptable salts, hydrates, solvates, polymorphs, or prodrugs thereof.

Sipoglitazar or TAK-654

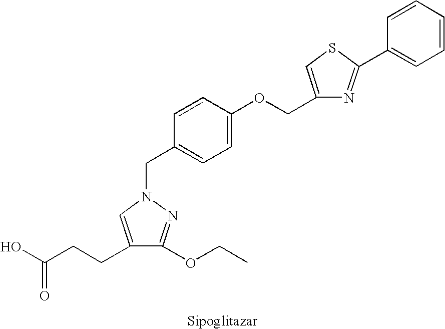

An embodiment of the invention is a method of treating neuropathic pain comprising treating a mammal in need of such treatment with a therapeutically effective amount of Sipoglitazar, or pharmaceutically acceptable salts, hydrates, solvates, polymorphs, or prodrugs thereof.

An embodiment of the invention is a method of treating neuropathic pain comprising treating a mammal in need of such treatment with a therapeutically effective amount of a compound of formula:

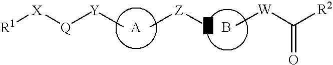

wherein

R¹ is an optionally substituted 5-membered heterocyclic group;

X is a bond, an oxygen atom, a sulfur atom, —CO—, —CS—, —CR³(OR⁴)— or —NR⁵— (R³ is a hydrogen atom or an optionally substituted hydrocarbon group, R⁴ is a hydrogen atom or a hydroxy-protecting group and R⁵ is a hydrogen atom, an optionally substituted hydrocarbon group or an amino-protecting group);

Q is a divalent hydrocarbon group having 1 to 20 carbon atoms;

Y is a bond, an oxygen atom, a sulfur atom, —SO—, —SO₂—, —NR⁶—, —CONR⁶— or —NR⁶CO— (R⁶ is a hydrogen atom or an optionally substituted hydrocarbon group);

ring A is an aromatic ring optionally further having 1 to 3 substituents;

Z is —(CH₂)n-Z¹— or —Z¹—(CH₂)n- (n is an integer of 0 to 8, Z¹ is a bond, an oxygen atom, a sulfur atom, —SO—, —SO₂—, —NR⁷—, —CONR⁷— or —NR⁷CO—(R⁷ is a hydrogen atom or an optionally substituted hydrocarbon group));

ring B is a 5-membered heterocycle optionally further having 1 to 3 substituents;

W is a divalent saturated hydrocarbon group having 1 to 20 carbon atoms; and

R² is —OR⁸ (R⁸ is a hydrogen atom or an optionally substituted hydrocarbon group) or —N R⁹R¹⁰ (R⁹ and R¹⁰ are the same or different and each is a hydrogen atom, an optionally substituted hydrocarbon group, an optionally substituted heterocyclic group, or an acyl group, or R⁹ and R¹⁰ may be linked to form an optionally substituted ring together with the adjacent nitrogen atom),

provided that, when ring B is a nitrogen-containing 5-membered heterocycle, then the nitrogen-containing 5-membered heterocycle does not have, on the ring-constituting N atom, a substituent represented by the formula:

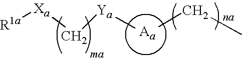

wherein

R^{1a} is an optionally substituted hydrocarbon group or an optionally substituted heterocyclic group;

Xa is a bond, an oxygen atom, a sulfur atom, —CO—, —CS—, —CR^{2a}(OR^{3a})— or —NR^{4a}— (R^{2a} and R^{4a} are each a hydrogen atom or an optionally substituted hydrocarbon group and R^{3a} is a hydrogen atom or a hydroxy-protecting group);

ma is an integer of 0 to 3;

Ya is an oxygen atom, a sulfur atom, —SO—, —SO₂—, —NR^{5a}—, CONR^{5a}— or —NR^{5a}CO—(R^{5a} is a hydrogen atom or an optionally substituted hydrocarbon group);

ring Aa is an aromatic ring optionally further having 1 to 3 substituents; and

na is an integer of 1 to 8,

or pharmaceutically acceptable salts, hydrates, solvates, polymorphs, or prodrugs thereof.

Experimental Results

Three models in rats have been shown to correlate well to clinical outcome both with respect to the rank order of active (Gabapentin, Pregabalin, Amitriptyline, Carbamazepine and N-type Ca++ blockers) and inactive (SSRI and NSAIDs) substances, and also between experimental and effective therapeutic doses. These models are based on three surgical procedures: (i) the spinal nerve ligation (SNL) [Kim, S. and J. Chung, An experimental model for peripheralneuropathy produced by segmental spinal nerve ligation in the rat. Pain, 1992. 50: p. 355-363.]; (ii) the partial sciatic nerve lesion (Seltzer) [Seltzer, Z., R. Dubner, and Y. Shir, A novel behavioral model of neuropathic pain disorders produced in rats by partial sciatic nerve injury. Pain, 1990. 43: p. 205-218.]; (iii) and the chronic constriction injury [Bennett, G. and Y. Xie, A peripheral mononeuropathy in rat that produces disorders of pain sensation like those seen in man. Pain, 1988. 33: p. 87-107.].

The ability of the compounds of Formulas I, II, III, IV, V, VI, VII, VIII and IX, including particularly Tesaglitazar, Muraglitazar, Peliglitazar, Farglitazar, Reglitazar, Naveglitazar, Oxeglitazar, Edaglitazone, Imiglitazar and Sipoglitazar, to treat neuropathic pain in mammals can be demonstrated using the SNL experimental protocol of Table 1.

TABLE 1
Experimental protocol.

| Day | Procedure | Drug | Notes |
|---|---|---|---|
| 0 | AM followed | None | Establish baseline behavior. |
| | by SNL | | Perform SNL. |
| 14 | AM | Vehicle | Confirm stable pain condition. |
| 15 | GPN followed | GPN 100 mg/kg IP | Comparator and positive control. |
| | by PWT | | |
| 16-20 | On each day, dose | PPARγ agonist at 100 mg/kg PO | Candidate drug effect. |
| | candidate drug | | |
| | followed by AM | | |
| 21 | GPN followed | GPN 100 mg/kg IP | Internal control to confirm any |
| | by AM | | apparent absence of effect for test |
| | | | compound. |

(AM, allodynia measurement by von Frey 1 h. post-drug or vehicle administration; GPN, gabapentin; IP, intraperitoneal.)

### Effect of Farglitazar on Mechanical Allodynia Induced by Spinal Nerve Ligation in Rats

Male Sprague-Dawley rats (Hsd:Sprague-Dawley®SD®, Harlan, Indianapolis, Ind., U.S.A.) weighing 223±2 g on Day14 were housed three per cage. Animals had free access to food and water and were maintained on a 12:12 h light/dark schedule for the entire duration of the study. The animal colony was maintained at 21° C. and 60% humidity. All experiments were conducted in accordance with the International Association for the Study of Pain guidelines and were approved by the University of Minnesota Animal Care and Use Committee.

The Spinal Nerve Ligation (SNL) model was used to induce chronic neuropathic pain. The animals were anesthetized with isoflurane, the left L6 transverse process was removed, and the L5 and L6 spinal nerves were tightly ligated with 6-0 silk suture. The wound was then closed with internal sutures and external staples.

Baseline, post-injury and post-treatment values for non-noxious mechanical sensitivity were evaluated using 8 Semmes-Weinstein filaments (Stoelting, Wood Dale, Ill., USA) with varying stiffness (0.4, 0.7, 1.2, 2.0, 3.6, 5.5, 8.5, and 15 g) according to the up-down method. Animals were placed on a perforated metallic platform and allowed to acclimate to their surroundings for a minimum of 30 minutes before testing. The mean and standard error of the mean (SEM) were determined for each paw in each treatment group. Since this stimulus is normally not considered painful, significant injury-induced increases in responsiveness in this test are interpreted as a measure of mechanical allodynia.

Statistical analyses were conducted using Prism™ 4.01 (GraphPad, San Diego, Calif., USA). Mechanical hypersensitivity of the injured paw was determined by comparing pre-SNL to post-SNL values at Day14. Data were analyzed using the Wilcoxon test. Effect of vehicle was tested by comparing post-SNL to post-vehicle values using the Wilcoxon test. Drug effect was analyzed by comparing post-vehicle and post-drug values using the Friedman test followed by a Dunn's post hoc test.

Farglitazar was dissolved in dimethyl sulfoxide (Sigma, cat. D8418, batch 105K00451) and diluted with 0.9% sterile saline (Baxter, cat. 2F7124, lot G046730) to the final concentration containing less than 2% dimethyl sulfoxide and ultrasound dispersed for five minutes. Farglitazar and vehicle were administered with a volume of 5 ml/kg.

Farglitazar 20 mg/kg PO significantly (p<0.05 vs. vehicle, Dunn's post hoc test) reduced mechanical allodynia on post-SNL day 16.

The dosage of a specific active compound of the invention depends upon many factors that are well known to those skilled in the art, for example, the particular compound; the condition being treated; the age, weight, and clinical condition of the recipient patient; and the experience and judgment of the clinician or practitioner administering the therapy. An effective amount of the compound is that which provides either subjective relief of symptoms or an objectively identifiable improvement as noted by the clinician or other qualified observer. The dosing range varies with the compound used, the route of administration and the potency of the particular compound. For example, the dosing ranges based on pre-clinical and clinical data described (above) would be Tesaglitazar 0.01-1 mg/kg, Muraglitazar 0.01-1 mg/kg, Peliglitazar 0.01-1 mg/kg, Farglitazar 0.03-3 mg/kg, Reglitazar 0.05-5 mg/kg, Naveglitazar 0.1-10 mg/kg, Oxeglitazar 1-100 mg/kg, Edaglitazone 0.01-1 mg/kg, Imiglitazar 0.1-10 mg/kg and Sipoglitazar 0.1-10 mg/kg.

### DEFINITIONS

The phrase “a” or “an” entity as used herein refers to one or more of that entity; for example, a compound refers to one or more compounds or at least one compound. As such, the terms “a” or (or “an”), “one or more”, and “at least one” can be used interchangeably herein.

The terms “optional” or “optionally” as used herein means that a subsequently described event or circumstance may but need not occur, and that the description includes instances where the event or circumstance occurs and instances in which it does not. For example, “optional bond” means that the bond may or may not be present, and that the description includes single, double, or triple bonds.

The term “independently” is used herein to indicate that a variable is applied in any one instance without regard to the presence or absence of a variable having that same or a different definition within the same compound. Thus, in a compound in which R appears twice and is defined as “independently carbon or nitrogen”, both R's can be Carbon, both R's can be nitrogen, or one R can be carbon and the other nitrogen.

The term “alkenyl” refers to an unsubstituted hydrocarbon chain radical having from 2 to 10 carbon atoms having one or two olefinic double bonds, preferably one olefinic double bond. The term “C_{2-N} alkenyl” refers to an alkenyl comprising 2 to N carbon atoms where N is an integer having the following values: 3, 4, 5, 6, 7, 8, 9, or 10. The term “C₂₋₁₀ alkenyl” refers to an alkenyl comprising 2 to 10 carbon atoms. Examples include, but are not limited to vinyl, 1-propenyl, 2-propenyl, (allyl) or 2-butenyl(crotyl).

The term “halogenated alkenyl” refers to an alkenyl comprising at least one of F, Cl, Br, and I.

The term alkyl refers to an unbranched or branched chain, saturated, monovalent hydrocarbon residue containing 1 to 30 carbon atoms. The term “C_{1-N} alkyl” refers to an alkyl comprising 1 to N carbon atoms, where N is an integer having the following values: 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. The term “C₁₋₄” alkyl refers to an alkyl contain 1 to 4 carbon atoms. The term “low alkyl” or “lower alkyl” denotes a straight or branched chain hydrocarbon residue comprising 1 to 8 carbon atoms. “C₁₋₂₀ alkyl” as used herein refers to an alkyl comprising 1 to 20 carbon atoms. “C₁₋₁₀ alkyl” as used herein refers to an alkyl comprising 1 to 10 carbon atoms. Examples of alkyl groups include, but are not limited to, methyl, ethyl, propyl, i-propyl, n-butyl, i-butyl, t-butyl, pentyl, isopentyl, neopentyl, hexyl, heptyl, and octyl. The term (ar)alkyl or (heteroaryl)alkyl indicate the alkyl group is optionally substituted by an aryl or a heteroaryl group respectively.

The term “halogenated alkyl” (or “haloalkyl”) refers to an unbranched or branched chain alkyl comprising at least one of F, Cl, Br, and I. The term “C₁₋₃ haloalkyl” refers to a haloalkyl comprising 1 to 3 carbons and at least one of F, Cl, Br, and I. The term “halogenated lower alkyl” refers to a haloalkyl comprising 1 to 8 carbon atoms and at least one of F, Cl, Br, and I. Examples include, but are not limited to, fluoromethyl, chloromethyl, bromomethyl, iodomethyl, difluoromethyl, dichloromethyl, dibromomethyl, diiodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chhoroethyl, 2-bromoethyl, 2-iodoethyl, 2,2-difluoroethyl, 2,2-dichloroethyl, 2,2-dibromoethyl, 2,2-diiodoethyl, 3-fluoropropyl, 3-chloropropyl, 3-bromopropyl, 3-iodopropyl, 2,2,2-trifluoroethyl, 1,1,2,2,2-pentafluoroethyl, 1-fluoro-1-chloroethyl, or 1-fluororo-1-chloro-1-bromoethyl.

The term “alkynyl” refers to an unbranched or branched hydrocarbon chain radical having from 2 to 10 carbon atoms, preferably 2 to 5 carbon atoms, and having one triple bond. The term “C_{2-N} alkynyl” refers to an alkynyl comprising 2 to N carbon atoms, where N is an integer having the following values: 2, 3, 4, 5, 6, 7, 8, 9, or 10. The term “C₂₋₄ alkynyl” refers to an alkynyl comprising 2 to 4 carbon atoms. The term “C₂₋₁₀ alkynyl” refers to an alkynyl comprising 2 to 10 carbon atoms. Examples include, but are not limited to, ethynyl, 1-propynyl, 2-propynyl, 1-butynyl, 2-butynyl, or 3-butynyl.

The term “halogenated alkynyl” refers to an unbranched or branched hydrocarbon chain radical having from 2 to 10 carbon atoms preferably 2 to 5 carbon atoms, and having one triple bond and at least one of F, Cl, Br, and I.

The term “cycloalkyl” refers to a saturated carbocyclic ring comprising 3 to 8 carbon atoms, i.e. cyclopropyl, cyclobutyl, cyclopentyl, cyclohexyl, cycloheptyl or cyclooctyl. The term “C₃₋₇ cycloalkyl” as used herein refers to a cycloalkyl comprising 3 to 7 carbons in the carbocyclic ring.

The term “alkoxy” refers to an —O-alkyl group, wherein alkyl is defined above. Examples include, but are not limited to, methoxy, ethoxy, n-propyloxy, i-propyloxy, n-butyloxy, i-butyloxy, t-butyloxy. “Lower alkoxy” or “low alkoxy” or “low alkoxyl” as used herein denotes an alkoxy group with a “lower alkyl” group as previously defined. “C₁₋₁₀ alkoxy” refers to an —O-alkyl wherein alkyl is C₁₋₁₀.

The term “halogenated alkoxy” refers to an —O-alkyl group in which the alkyl group comprises at least one of F, Cl, Br, and I.

The term “halogenated lower alkoxy” or “halogenated low alkoxy” refers to an —O-(lower alkyl) group in which the lower alkyl group comprises at least one of F, Cl, Br, and I.

The term “substituted”, as used herein, means that one or more hydrogens on the designated atom is replaced with a selection from the indicated group, provided that the designated atom's normal valency is not exceeded, and that the substitution results in a stable compound.

The term “protected”, as used herein and unless otherwise defined, refers to a group that is added to an oxygen, nitrogen, or phosphorus atom to prevent its further reaction or for other purposes. A wide variety of oxygen and nitrogen protecting groups are known to those skilled in the art of organic synthesis. Non-limiting examples include: C(O)-alkyl, C(O)Ph, C(O)aryl, CH₃, CH₂-alkyl, CH₂-alkenyl, CH₂Ph, CH₂-aryl, CH₂O-alkyl, CH₂O-aryl, SO₂-alkyl, SO₂-aryl, tert-butyldimethylsilyl, tert-butyldiphenylsilyl, and 1,3-(1,1,3,3-tetraisopropyldisiloxanylidene).

The term “halo” or as used herein includes fluoro, chloro, bromo, and iodo.

The term “pharmaceutically acceptable salt or prodrug” is used throughout the specification to describe any pharmaceutically acceptable form (such as an ester, phosphate ester, salt of an ester or related group) of a compound which upon administration to a mammal, provides the active compound. Pharmaceutically acceptable salts include those derived from pharmaceutically acceptable inorganic or organic bases and acids. Pharmaceutically acceptable prodrugs refer to a compound that is metabolized, for example hydrolyzed or oxidized, in the host to form a compound of a method of the present invention. A “pharmaceutically acceptable salt” form of an active ingredient may also initially confer a desirable pharmacokinetic property on the active ingredient which was absent in the non-salt form, and may even positively affect the pharmacodynamics of the active ingredient with respect to its therapeutic activity in the body. The phrase “pharmaceutically acceptable salt” of a compound as used herein means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as glycolic acid, pyruvic acid, lactic acid, malonic acid, maleic acid, fumaric acid, tartaric acid, citric acid, 3-(4-hydroxybenzoyl)benzoic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, lauryl sulfuric acid, gluconic acid, glutamic acid, salicyclic acid, muconic acid, and the like or (2) basic addition salts formed with the conjugate bases of any of the inorganic acids listed above, wherein the conjugate bases comprise a cationic component selected from among Na⁺, K⁺, Mg2⁺, Ca2⁺, NHgR′″4-g⁺, in which R′″ is a C₁₋₃ alkyl and g is a number selected from among 0, 1, 2, 3, or 4. It should be understood that all references to pharmaceutically acceptable salts include solvent addition forms (solvates), water addition forms (hydrates), or crystal forms (polymorphs) as defined herein, of the same acid additions salts.

Any of the compounds described herein can be administered as a prodrug to increase the activity, bioavailability, stability or otherwise alter the properties of the selected compound. A number of prodrug ligands are known.

The term “host” or “subject” as used herein, refers to a unicellular or multicellular organism in which the virus can replicate, including but not limited to cell lines and animals, and preferably a human. Alternatively, the host can be carrying a part of the viral genome, whose replication or function can be altered by the compounds of the present invention. The term host specifically refers to infected cells, cells transfected with all or part of the viral genome and animals.

The compounds used in methods of the present invention may be formulated in a wide variety of oral administration dosage forms and carriers. Oral administration can be in the form of tablets, coated tablets, hard and soft gelatin capsules, solutions, emulsions, syrups, or suspensions. Compounds used in methods of the present invention are efficacious when administered by suppository administration, among other routes of administration. The most convenient manner of administration is generally oral using a convenient daily dosing regimen which can be adjusted according to the severity of the disease and the patient's response to the antiviral medication.

A compound or compounds used in methods of the present invention, as well as their pharmaceutically acceptable salts, solvates, hydrates, prodrugs, and polymorphs, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations or liquids such as suspensions, emulsions, or filled capsules for oral use; or in the form of suppositories for rectal or vaginal administration. A typical preparation will contain from about 5% to about 95% active compound or compounds (w/w). The term “preparation or “dosage form” is intended to include both solid and liquid formulations of the active compound and one skilled in the art will appreciate that an active ingredient can exist in different preparations depending on the desired dose and pharmacokinetic parameters.

The term “excipient” as used herein refers to a compound that is used to prepare a pharmaceutical composition, and is generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

Solid form preparations include powders, tablets, pills capsules, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component colorants, flavors, stabilizers, buffers, artificial and natural sweeteners, dispersants, thickeners, solubilizing agents, and the like.

Liquid formulations also are suitable for oral administration include liquid formulations including emulsions, syrups, elixirs and aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous suspensions can be prepared by dispersing the finely divided active component in water with viscous material, such as natural or synthetic gums, resins, methylcellulose, sodium carboxymethylcellulose, and other well known suspending agents.

The compounds used in methods of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool and to solidify.

The compounds used in methods of the present invention may be formulated for vaginal administration. Pessaries, tampons, creams, gels, pastes, foams or sprays containing in addition to the active ingredient such carriers as are known in the art to be appropriate.

Suitable formulations along with pharmaceutical carriers, diluents and excipients are described in Remington: The Science and Practice of Pharmacy 1995, edited by E. W. Martin, Mack Publishing Company, 19th Edition, Easton, Pa., which is hereby incorporated by reference. A skilled formulation scientist may modify the formulations within the teachings of the specification to provide numerous formulations for a particular route of administration without rendering the compositions of the present invention unstable or comprising their therapeutic activity.

The modification of the present compounds to render them more soluble in water or other vehicle, for example, may be easily accomplished by minor modifications (e.g., salt formulation), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “medicament” means a substance used in a method of treatment and/or prophylaxis of a subject in need thereof, wherein the substance includes, but is not limited to, a composition, a formulation, a dosage from, and the like, comprising a compound of formula I. It is contemplated that the use of a compound of a method of the invention in the manufacture of a medicament for the treatment of any of the conditions disclosed herein can be any of the compounds contemplated in any of the aspects of the invention, either alone or in combination with other compounds of the methods of the present invention.

The term “therapeutically effective amount” as used herein means an amount required to reduce symptoms of neuropathic pain in an individual. The dose will be adjusted to the individual requirements in each particular case. That dosage can vary within wide limits depending upon numerous factors such as the severity of the condition to be treated, the age and general health condition of the patient, other medicaments with which the patient is being treated, the route and form of administration and the preferences and experience of the medical practitioner involved. For oral administration, a daily dosage of between about 0.1 and about 10 g, including all values in between, such as 0.25, 0.5, 0.75, 1, 1.5, 2, 2.5, 3, 3.5, 4, 4.5, 5, 5.5, 6, 6.5, 7, 7.5, 8, 8.5, 9, and 9.5, per day should be appropriate in monotherapy and/or in combination therapy. A preferred daily dosage is between about 0.5 and about 7.5 g per day, a more preferred dosage is between 1.5 and about 6.0 g per day. One of ordinary skill in treating conditions described herein will be able, without undue experimentation and in reliance on personal knowledge, experience, and the disclosures of this application, to ascertain a therapeutically effective amount of the compounds of the methods of the present invention for a given condition and patient.

1. A method of treating neuropathic pain, comprising administering a pharmaceutical composition to a mammal in need of such treatment, wherein the pharmaceutical composition comprises a therapeutically effective amount of an agonist of PPARγ.

2. The method of treating neuropathic pain of claim 1, wherein the mammal is a human.

3. The method of treating neuropathic pain of claim 2, wherein the agonist of PPARγ is Tesaglitazar 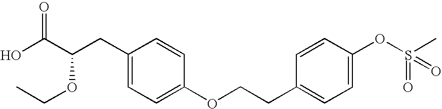
or a pharmaceutically acceptable salt, solvate, ester or hydrate thereof.

4. The method of treating neuropathic pain of claim 2, wherein the agonist of PPARγ is a compound of Formula II: 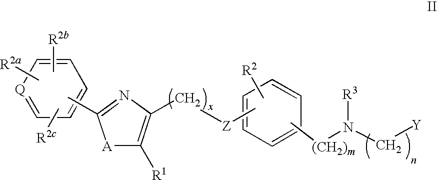
wherein:
x is 1, 2, 3, or 4;
m is 1 or 2;
n is 1 or 2;
Q is C or N;
A is O or S;
Z is O or a bond;
R¹ is H or alkyl;
X is CH or N;
R² is H, alkyl, alkoxy, halogen amino, or substituted amino;
R^{2a}, R^{2b}, and R^{2c} are independently H, alkyl, alkoxy, halogen, amino, or substituted amino;
R³ is H, alkyl, arylalkyl, aryloxycarbonyl, alkyloxycarbonyl, alkynyloxycarbonyl, alkenyloxycarbonyl, arylcarbonyl, alkylcarbonyl, aryl, heteroaryl, alkyl(halo)aryloxycarbonyl, alkyloxy(halo)aryloxy-carbonyl, cycloalkylaryloxycarbonyl, cycloalkyloxyaryloxycarbonyl, cycloheteroalkyl, heteroarylcarbonyl, heteroaryl-heteroarylalkyl, alkylcarbonylamino, arylcarbonylamino, heteroarylcarbonylamino, alkoxycarbonylamino, aryloxycarbonylamino, heteroaryloxycarbonylamino, heteroaryl-heteroarylcarbonyl, alkylsulfonyl, alkenylsulfonyl, heteroaryloxycarbonyl, cycloheteroalkyloxycarbonyl, heteroarylalkyl, aminocarbonyl, substituted aminocarbonyl, alkylaminocarbonyl, arylaminocarbonyl, heteroarylalkenyl, cycloheteroalkyl-heteroarylalkyl; hydroxyalkyl, alkoxy, alkoxyaryloxycarbonyl, arylalkyloxycarbonyl, alkylaryloxycarbonyl, arylheteroarylalkyl, arylalkylarylalkyl, aryloxyarylalkyl, haloalkoxyaryloxycarbonyl, alkoxycarbonylaryloxycarbonyl, aryloxyaryloxycarbonyl, arylsulfinylarylcarbonyl, arylthioarylcarbonyl, alkoxycarbonylaryloxycarbonyl, arylalkenyloxycarbonyl, heteroaryloxyarylalkyl, aryloxyarylcarbonyl, aryloxyarylalkyloxycarbonyl, arylalkenyloxycarbonyl, arylalkylcarbonyl, aryloxyalkyloxycarbonyl, arylalkylsulfonyl, arylthiocarbonyl, arylalkenylsulfonyl, heteroarylsulfonyl, arylsulfonyl, alkoxyarylalkyl, heteroarylalkoxycarbonyl, arylheteroarylalkyl, alkoxyarylcarbonyl, aryloxyheteroarylalkyl, heteroarylalkyloxyarylalkyl, arylarylalkyl, arylalkenylarylalkyl, arylalkoxyarylalkyl, arylcarbonylarylalkyl, alkylaryloxyarylalkyl, arylalkoxycarbonylheteroarylalkyl, heteroarylarylalkyl, arylcarbonylheteroarylalkyl, heteroaryloxyarylalkyl, arylalkenylheteroarylalkyl, arylaminoarylalkyl, aminocarbonylarylarylalkyl;
Y is CO₂R⁴ (where R⁴ is H or alkyl, or a prodrug ester) or Y is a C-linked 1-tetrazole, a phosphinic acid of the structure P(O)(OR^{4a})R⁵, (where R^{4a} ia H or a prodrug ester, R⁵ is alkyl or aryl) or phosphonic acid of the structure P(O)(OR^{4a})₂, (where R^{4a} is H or a prodrug ester);
(CH₂)ₓ, (CH₂)ₙ, and (CH₂)ₘ may be optionally substituted with 1, 2, or 3 substituents;
including stereoisomers thereof, prodrug esters thereof, and pharmaceutically acceptable salts, hydrates, solvates, prodrugs, and polymorphs thereof, with the proviso that
where X is CH, A ia O, Q is C, Z is O, and Y is CO₂R⁴, then R³ is other than H or alkyl containing 1 to 5 carbons in the normal chain;
or a pharmaceutically acceptable salt, hydrate, solvate or prodrug thereof.

5. The method of treating neuropathic pain of claim 4, wherein the agonist of PPARγ is Muraglitazar 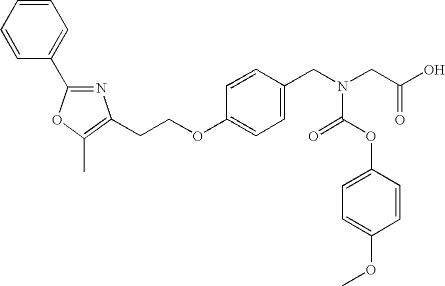
or a pharmaceutically acceptable salt, hydrate, solvate or prodrug thereof.

6. The method of treating neuropathic pain of claim 4, wherein the agonist of PPARγ is Peliglitazar 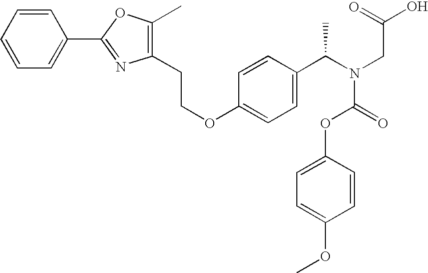
or a pharmaceutically acceptable salt, hydrate, solvate or prodrug thereof.

7. The method of treating neuropathic pain of claim 2, wherein the agonist of PPARγ is a compound of Formula III: 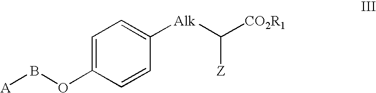
wherein
A is selected from the group consisting of:
(i) phenyl, wherein the phenyl is optionally substituted by one or more of the following groups: halogen atoms, C₁₋₆alkyl, C₁₋₃ alkoxy, C₁₋₃ fluoroalkoxy, nitrile, or —NR⁷R⁸ where R⁷ and R⁸ are independently hydrogen or C₁₋₃ alkyl;
(ii) a 5- or 6-membered heterocyclic group containing at least one heteroatom selected from oxygen, nitrogen and sulfur; and
(iii) a fused bicyclic ring 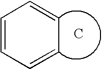
wherein ring C represents a heterocyclic group as defined in point (ii) above, which bicyclic ring is attached to group B via a ring atom of C;
B is selected from the group consisting of:
(iv) C₁₋₆ alkene;
(v) -MC₁₋₆ alkene or C₁₋₆ alkeneMC₁₋₆ alkene, wherein M is O, S, or —NR² wherein R² represents hydrogen or C₁₋₃ alkyl;
(vi) a 5- or 6-membered heterocyclic group containing at least one nitrogen heteroatom and optionally at least one further heteroatom selected from oxygen, nitrogen and sulfur and optionally substituted by C₁₋₃ alkyl; and
(vii) Het-C₁₋₆alkylene, wherein Het represents a heterocyclic group as defined in point (vi) above;
Alk represents C₁₋₃ alkylene;
R₁ represents hydrogen or C₁₋₃ alkyl;
Z is selected from the group consisting of:
(viii) —(C₁₋₃alkylene) phenyl, which phenyl is optionally substituted by one or more halogen atoms; and
(ix) —NR³R⁴, wherein R³ represents hydrogen or C₁₋₃alkyl, and R⁴ represents —Y—(CH(OH)-T-R⁵, or —Y—(CH(OH))-T-R⁵, wherein:
(a) Y represents a bond, C₁₋₆ alkylene, C₂₋₆alkenylene, C₄₋₆ cycloalkene or cycloalkenylene, a heterocyclic group as defined in point (vi) above, or phenyl optionally substituted by one or more C₁₋₃ alkyl groups and/or one or more halogen atoms;
(b) T represents a bond, C₁₋₃ alkyleneoxy, —O— or —N(R⁶)—, wherein R⁶ represents hydrogen or C₁₋₃ alkyl;
(c) R⁵ represents C₁₋₆ alkyl, C₄₋₆ cycloalkyl or cycloalkenyl, phenyl (optionally substituted by one or more of the following groups; halogen atoms, C₁₋₃ alkyl, C₁₋₃ alkoxy groups, C₀₋₃ alkyleneNR⁹R¹⁰ (where each R⁹ and R¹⁰ is independently hydrogen, C₁₋₃ alkyl, —SO₂C₁₋₃alkyl, or —CO₂C₁₋₃alkyl, —SO₂NHC₁₋₃alkyl), C₀₋₃ alkyleneCO₂H, C₀₋₃alkyleneCO₂C₁₋₃alkyl, or —OCO₂C(O)NH₂), a 5- or 6-membered heterocyclic group as defined in point (ii) above, a bicyclic fused ring 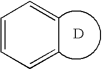
wherein ring D represents a 5- or 6-membered heterocyclic group containing at least one heteroatom selected from oxygen, nitrogen and sulfur and optionally substituted by (═O), which bicyclic ring is attach to T vi a ring atom of ring D: or —C₁₋₆ alkyleneMR¹¹; M is O, S, or NR¹² wherein R¹² and R¹¹ are independently hydrogen or C₁₋₃ alkyl;
or a tautomeric form or a pharmaceutically acceptable salt, hydrate, solvate or prodrug thereof.

8. The method of treating neuropathic pain of claim 7, wherein the agonist of PPARγ is Farglitazar 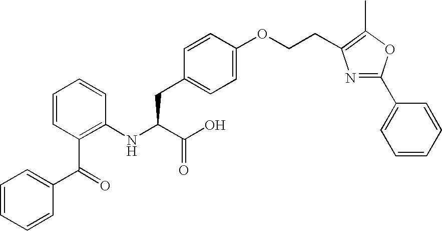
or a pharmaceutically acceptable salt, hydrate, solvate or prodrug thereof.

9. The method of treating neuropathic pain of claim 2, wherein the agonist of PPARγ is a compound of Formula IV: 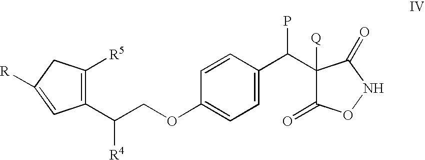
wherein:
R is an optionally substituted aromatic hydrocarbon, an optionally substituted alicyclic hydrocarbon, an optionally substituted heterocyclic group, an optionally substituted condensed heterocyclic group or a group of the formula: 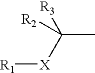
wherein R₁ is an optionally substituted aromatic hydrocarbon, an optionally substituted alicyclic hydrocarbon, an optionally substituted heterocyclic group or an optionally substituted condensed heterocyclic group, R₂ and R₃ are the same or different and each is a hydrogen atom or a lower alkyl, and X is an oxygen atom, a sulfur atom or a secondary amino;
R⁴ is a hydrogen atom, a lower alkyl or a hydroxy;
R⁵ is a lower alkyl optionally substituted by hydroxy; and
P and Q are each a hydrogen atom or P and Q together form a bond;
or a pharmaceutically acceptable salt, hydrate, solvate, ester or prodrug thereof.

10. The method of treating neuropathic pain of claim 9, wherein the agonist of PPARγ is Reglitazar 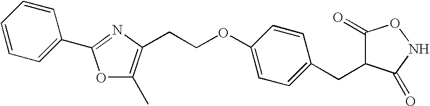
or a pharmaceutically acceptable salt, solvate, ester, hydrate or prodrug thereof.

11. The method of treating neuropathic pain of claim 2, wherein the agonist of PPARγ is a compound of Formula V: 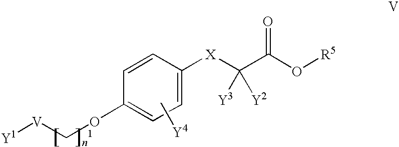
n¹ is 2, 3, 4 or 5;
V is a bond or O;
X is CH₂ or O;
p is 0 or 1;
m is 1-4;
Y¹ is: 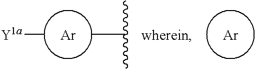
is aryl or heteroaryl optionally substituted with one or more groups independently selected from the group consisting of hydrogen, C₁₋₆ alkyl, C₁₋₆ alkoxy, halo, haloalkyl and haloalkyloxy;
Y^{1a} is: hydrogen, (C₀₋₃)alkyl-aryl, C(O)-aryl, heteroaryl, cycloalkyl, heterocycloalkyl, aryloxy, NR⁵(CH₂)ₘOR⁵, aryl-Z-aryl, aryl-Z-heteroaryl, aryl-Z-cycloalkyl, aryl-Z-heterocycloalkyl, heteroaryl-Z-aryl, heteroaryl-Z-heterocycloalkyl or heterocycloalkyl-Z-aryl,
wherein aryl, cycloalkyl, aryloxy, heteroaryl, and heterocycloalkyl are optionally substituted with one or more substituents independently selected from the group consisting of:
halo, hydroxyl, nitro, cyano, C₁₋₆ alkyl, C₁₋₆ alkoxy optionally substituted with N(R⁵)₂, haloalkyl, N(R⁵)₂, N[C(O)R⁵]₂, N[S(O)₂R⁵]₂, NR⁵S(O)₂R⁵, NR⁵C(O)R⁵, NR⁵C(O)OR⁵, C(O)N(R⁵)₂, C(O)OR⁵ and C(O)R⁵;
Z is: a bond, -oxygen-, —C(O)NR⁵—, —NR⁵C(O)—, —NR⁵C(O)O—, —C(O)—, —NR⁵, —[O]p(CH₂)m—, —(CH₂)m[O]p—, —NR⁵(CH₂)m- or —(CH₂)mNR⁵—;
Y² and Y³ are each independently: hydrogen, C₁₋₆alkyl or C₁₋₆ alkoxy;
Y⁴ is: (C₁₋₃)alkyl-NR⁵C(O)—(C₀₋₅)alkyl-Y⁷—, (C₁₋₃)alkyl-NR⁵C(O)—(C₂₋₅)alkenyl-Y⁷, (C₁₋₃)alkyl-NR⁵C(O)—(C₂₋₅)alkynyl-Y⁷; (C₁₋₃)alkyl-NR⁵C(O)O—(C₀₋₅)alkyl-(C₁₋₃)alkyl-NR⁵C(O)NR⁵—(C₀₋₅)alkyl-(C₁₋₃)alkyl-NR⁵C(S)NR⁵—(C₀₋₅)alkyl-(C₀₋₃)alkyl-C(O)NR⁵—(C₀₋₅)alkyl-Y⁷, (C₀₋₃)alkyl-OC(O)NY¹⁰Y¹¹, (C₁₋₃)alkyl-NY¹⁰Y¹¹, (C₁₋₃)alkyl-O—(C₀₋₅)alkyl-Y⁷, (C₁₋₃)alkyl-S—(C₀₋₅)alkyl-Y⁷ or CN;
Y⁷ is: hydrogen, aryl, heteroaryl, C₁₋₁₂ alkyl, C₁₋₆ alkoxy, cycloalkyl, heterocycloalkyl, aryloxy, C(O)-heteroaryl or SR⁶,
wherein alkyl, aryl, aryloxy, alkoxy, heteroaryl, cycloalkyl, and heterocycloalkyl are optionally substituted with one or more groups independently selected from R⁷:
Y¹⁰ and Y¹¹ are each independently: hydrogen, aryl, heteroaryl, C₁₋₁₀alkyl, cycloalkyl, SO₂ (R⁶);
or
Y¹⁰ and Y¹¹ together are a 5- to 10-membered heterocycloalkyl ring or heterocycloalkyl ring fused with aryl, and the heterocycloalkyl ring optionally containing one or more heteroatoms selected from N, O or S; and wherein, aryl, heteroaryl, heterocycloalkyl and alkyl are optionally substituted with one or more substituents independently selected from R⁷;
R⁵ is: hydrogen or C₁₋₆ alkyl;
R⁶ is: hydrogen, C₁₋₁₀ alkyl, cycloalkyl, aryl, or heteroaryl, wherein alkyl, cycloalkyl, aryl and heteroaryl are optionally substituted with one or more substituents independently selected from R⁷; R⁷ is: halo, nitro, oxo, cyano, hydroxyl, benzyl, phenyl, phenoxy, heteroaryl, C(O)R⁶, C₁₋₁₀ alkyl, C₁₋₆ alkoxy, C₁₋₆ haloalkyl, C₁₋₆ haloalkyloxy, O(CH₂)m-phenyl, (CH₂)mOC(O)-aryl, C(O)OR⁵, S(O)₂R⁵, S(O)₂N(R⁵)₂, SR⁵ or N(R⁵)₂, wherein phenyl and phenoxy are optionally substituted with one or more groups independently selected from halo or trifluoromethyl;
or a pharmaceutically acceptable salt, hydrate, solvate, ester or prodrug thereof.

12. The method of treating neuropathic pain of claim 11, wherein the agonist of PPARγ is Naveglitazar 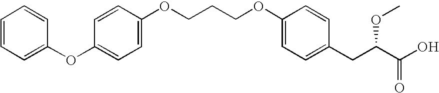
or a pharmaceutically acceptable salt, solvate, ester, hydrate or prodrug thereof.

13. The method of treating neuropathic pain of claim 2, wherein the agonist of PPARγ is a compound of Formula VI: 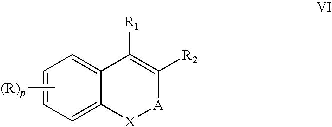
wherein:
X represents O or S;
A represents either the divalent radical —(CH₂)ₛ—CO—(CH₂)ₜ— or the divalent radical —(CH₂)ₛ—CR₃R₄—(CH₂)ₜ— in which radicals s=t=0 or else one of s and t has the value 0 and the other has the value 1;
R₄ represents a hydrogen atom or a (C₁-C₁₅)alkyl group;
R₁ and R₂ independently represent the Z chain defined below; a hydrogen atom; a (C₁-C₁₈)alkyl group; a (C₂-C₁₈)alkenyl group; a (C₂-C₁₈)alkynyl group; a (C₆-C₁₀)aryl group optionally substituted by a halogen atom, by an optionally halogenated (C₁-C₅)alkyl group or by an optionally halogenated (C₁-C₅)alkoxy group; or a mono- or bicyclic (C₄-C₁₂)heteroaryl group comprising one or more heteroatoms chosen from O, N and S which is optionally substituted by a halogen atom, by an optionally, halogenated (C₁-C₅)alkyl group or by an optionally halogenated (C₁-C₅)alkoxy group;
R₃ takes any one of meanings given above for R₁ and R₂, with the exception of the Z chain; or else
R₃ and R₄ together form a (C₂-C₆)alkylene chain optionally substituted by a halogen atom or by optionally halogenated (C₁-C₅)alkoxy;
R is chosen from a halogen atom; a cyano group; a nitro group; a carboxy group; an optionally halogenated (C₁-C₁₈)alkoxycarbonyl group; an Rₐ—CO—NH— or RₐR_{b} N—CO— group [in which Rₐ and R_{b} independently represent optionally halogenated (C₁-C₁₈)alkyl; a hydrogen atom; (C₆-C₁₀)aryl or (C₆-C₁₀)aryl(C₁-C₅)alkyl (where the aryl parts are optionally substituted by a halogen atom, by an optionally halogenated (C₁-C₅)alkyl group or by an optionally halogenated (C₁-C₅)alkoxy group); (C₃-C₁₂)cycloalkyl optionally substituted by a halogen atom, by an optionally halogenated (C₁-C₅)alkyl group or by an optionally halogenated (C₁-Cₛ)alkoxy group]; an optionally halogenated (C₁-C₁₈)alkyl group; optionally halogenated (C₁-C₁₈)alkoxy; and (C₆-C₁₀)aryl, (C₆-C₁₀)aryl(C₁-C₅)alkyl, (C₆-C₁₀)aryloxy, (C₃-C₁₂)cycloalkyl, (C₃-C₁₂)cycloalkenyl, (C₃-C₁₂)cycloalkyloxy, (C₃-C₁₂)cycloalkenyloxy or (C₆-C₁₀)aryloxycarbonyl in which the aryl, cycloalkyl and cycloalkenyl parts are optionally substituted by a halogen atom, by optionally halogenated (C₁-C₅)alkyl or by optionally halogenated (C₁-C₅)alkoxy;
p represents 0, 1, 2, 3 or 4;
Z represents the radical: 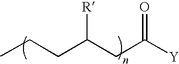
where n is 1 or 2;
the R′ groups independently represent a hydrogen atom; a (C₁-C₅)alkyl group; a (C₆-C₁₀)aryl group optionally substituted by a halogen atom, by an optionally halogenated (C₁-C₅)alkyl group or by optionally halogenated (C₁-C₅)alkoxy; or a mono- or bicyclic (C₄-C₁₂)heteroaryl group comprising one or more heteroatoms chosen from O, N and S which is optionally substituted by a halogen atom, by an optionally halogenated (C₁-C₅)alkyl group or by an optionally halogenated (C₁-C₅)alkoxy group;
Y represents —OH; (C₁-C₅)alkoxy; or the —NR_{c}, R_{d} group (in which R_{c} and R_{d} independently represent a hydrogen atom; (C₁-C₅)alkyl; (C₃-C₈)cycloalkyl optionally substituted by a halogen atom, by optionally halogenated (C₁-C₅)alkyl or by optionally halogenated (C₁-C₅)alkoxy; (C₆-C₁₀)aryl optionally substituted by a halogen atom, by optionally halogenated (C₁-C₅)alkyl or by optionally halogenated (C₁-C₅)alkoxy; it being understood that one and one alone from R₁ and R₂ represents the Z chain;
or a pharmaceutically acceptable salt, hydrate, solvate, ester or prodrug thereof.

14. The method of treating neuropathic pain of claim 13, wherein the agonist of PPARγ is Oxeglitazar 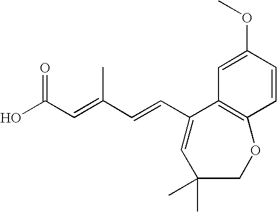
or a pharmaceutically acceptable salt, solvate, ester, hydrate or prodrug thereof.

15. The method of treating neuropathic pain of claim 2, wherein the agonist of PPARγ is a compound of Formula VII: 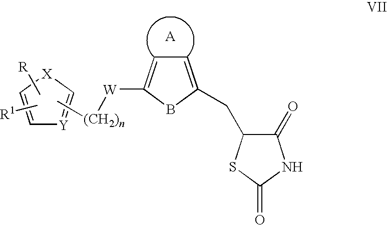
wherein:
A is a carbocyclic ring with 5 or 6 carbon atoms or a heterocyclic ring with a maximum of 4 heteroatoms in which the heteroatoms can be the same or different and denote oxygen, nitrogen, or sulfur and the heterocycles can if desired, carry an oxygen atom on one or several nitrogen atoms;
B is —CH═CH—, —N═CH—, —CH═N—, O, or S;
W is CH2, OCH(OH), CO or —CH═CH—;
X is S, O, or NR2 in which the residue R2 is hydrogen or C1-6 alkyl;
Y is CH or N;
R is naphthyl, pyridyl, furyl, thienyl, or phenyl which if desired is mono- or disubstituted with C1-3 alkyl, CF3, C1-3 alkoxy, F, Cl, or Br;
R1 is hydrogen or C1-6 alkyl; and
n is 1 to 3;
or a pharmaceutically acceptable salt, hydrate, solvate, ester or prodrug thereof.

16. The method of treating neuropathic pain of claim 15, wherein the agonist of PPARγ is Edaglitazone 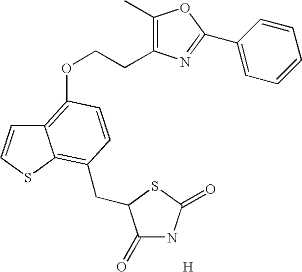
or a pharmaceutically acceptable salt, solvate, hydrate or prodrug thereof.

17. The method of treating neuropathic pain of claim 2, wherein the agonist of PPARγ is a compound of Formula VIII: 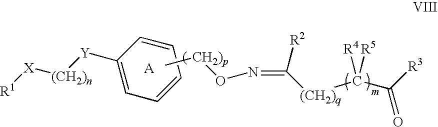
wherein:
R¹ is an optionally substituted hydrocarbon group, optionally substituted cyclic hydrocarbon group, or an optionally substituted heterocyclic group;
X is a bond, —CO—, —CH(OH)— or a group represented by —NR⁶— wherein R⁶ is a hydrogen atom or an optionally substituted alkyl group;
n is an integer of 1 to 3;
Y is an oxygen atom, a sulfur atom, —SO—, —SO₂— or a group represented by —NR⁷— wherein R⁷ is a hydrogen atom or an optionally substituted alkyl group;
a ring A is a benzene ring optionally having additional one to three substituents;
p is an integer of 1 to 8;
R² is a hydrogen atom, an optionally substituted hydrocarbon group or an optionally substituted heterocyclic group;
q is an integer of 0 to 6;
m is 0 or 1;
R³ is a hydroxy group, OR⁸ (R⁸ is an optionally substituted hydrocarbon group.) or NR⁹R¹⁰ (R⁹ and R¹⁰ are the same or different groups which are selected from a hydrogen atom, an optionally substituted hydrocarbon group, an optionally substituted heterocyclic group or an optionally substituted acyl group or R⁹ and R¹⁰ combine together to form a ring); R⁴ and R⁵ are the same or different groups which are selected from a hydrogen atom or an optionally substituted hydrocarbon group wherein R⁴ may form a ring with R²;
provided that when R¹ is a ethoxymethyl, a C₁₋₃ alkyl, phenyl or p-methoxyphenyl and q=m=O, R³ is NR⁹R¹⁰;
and provided that O-[2-chloro-4-(2-quinolylmethoxy)phenylmethyl]oxime of methylpyruvate and [2-chloro-4-(2-quinolylmethoxy)phenylmethyl]-2-iminoxy-propionic acid are excluded;
or a pharmaceutically acceptable salt, solvate, ester, hydrate or prodrug thereof.

18. The method of treating neuropathic pain of claim 17, wherein the agonist of PPARγ is Imiglitazar 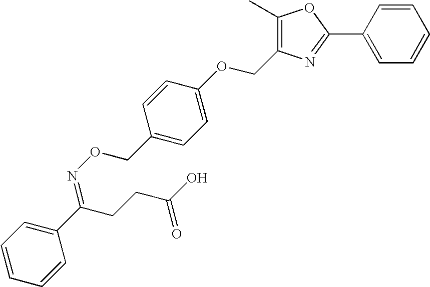
or a pharmaceutically acceptable salt, solvate, ester, hydrate or prodrug thereof.

19. The method of treating neuropathic pain of claim 2, wherein the agonist of PPARγ is a compound of Formula IX: 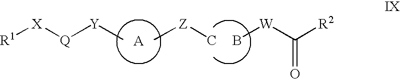
wherein
R¹ is an optionally substituted 5-membered heterocyclic group;
X is a bond, an oxygen atom, a sulfur atom, —CO—, —CS—, —CR³(OR⁴)— or —NR⁵— (R³ is a hydrogen atom or an optionally substituted hydrocarbon group, R⁴ is a hydrogen atom or a hydroxy-protecting group and R⁵ is a hydrogen atom, an optionally substituted hydrocarbon group or an amino-protecting group);
Q is a divalent hydrocarbon group having 1 to 20 carbon atoms;
Y is a bond, an oxygen atom, a sulfur atom, —SO—, —SO₂—, —NR⁶—, —CONR⁶— or —NR⁶CO— (R⁶ is a hydrogen atom or an optionally substituted hydrocarbon group);
ring A is an aromatic ring optionally further having 1 to 3 substituents;
Z is —(CH₂)n-Z¹— or —Z¹—(CH₂)n- (n is an integer of 0 to 8, Z¹ is a bond, an oxygen atom, a sulfur atom, —SO—, —SO₂—, —NR⁷—, —CONR⁷— or —NR⁷CO— (R⁷ is a hydrogen atom or an optionally substituted hydrocarbon group));
ring B is a 5-membered heterocycle optionally further having 1 to 3 substituents;
W is a divalent saturated hydrocarbon group having 1 to 20 carbon atoms; and
R² is —OR⁸ (R⁸ is a hydrogen atom or an optionally substituted hydrocarbon group) or —NR⁹R¹⁰(R⁹ and R¹⁰ are the same or different and each is a hydrogen atom, an optionally substituted hydrocarbon group, an optionally substituted heterocyclic group, or an acyl group, or R⁹ and R¹⁰ may be linked to form an optionally substituted ring together with the adjacent nitrogen atom),
provided that, when ring B is a nitrogen-containing 5-membered heterocycle, then the nitrogen-containing 5-membered heterocycle does not have, on the ring-constituting N atom, a substituent represented by the formula: 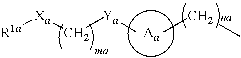
wherein
R^{1a} is an optionally substituted hydrocarbon group or an optionally substituted heterocyclic group;
Xa is a bond, an oxygen atom, a sulfur atom, —CO—, —CS—, —CR^{2a} (OR^{3a})— or —NR^{4a}— (R^{2a} and R^{4a} are each a hydrogen atom or an optionally substituted hydrocarbon group and R^{ia} is a hydrogen atom or a hydroxy-protecting group);
ma is an integer of 0 to 3;
Ya is an oxygen atom, a sulfur atom, —SO—, —SO₂—, CONR^{5a}— or —NR^{5a}CO— (R^{5a} is a hydrogen atom or an optionally substituted hydrocarbon group);
ring Aa is an aromatic ring optionally further having 1 to 3 substituents; and
na is an integer of 1 to 8;
or a pharmaceutically acceptable salt, solvate, ester, hydrate or prodrug thereof.

20. The method of treating neuropathic pain of claim 19, wherein the agonist of PPARγ is Sipoglitazar 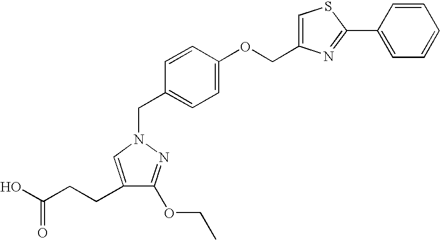
or a pharmaceutically acceptable salt, solvate, ester, hydrate or prodrug thereof.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)
